(19) United States
(12) Patent Application Publication
(10) Pub. No.: US 2007/0118176 A1
(43) Pub. Date: May 24, 2007

(54) Radiopaque bioabsorbable occluder

(76) Inventors: Steven W. Opolski, Carlisle, MA (US); Stephanie M. Kladakis, Watertown, MA (US)

Correspondence Address: WILMER CUTLER PICKERING HALE AND DORR LLP, 60 STATE STREET, BOSTON, MA, 02109, US

(21) Appl. No.: 11/585,395

(22) Filed: Oct. 24, 2006

Related U.S. Application Data
(60) Provisional application No. 60/729,549, filed on Oct. 24, 2005.

(51) Int. Cl. A61B 17/08

(52) U.S. Cl. 606/213

(57) ABSTRACT

The present invention provides an occluder for a biological defect, such as an atrial septal defect (ASD) or a patent foramen ovale (PFO). The occluder is at least partially formed of a radiopaque, bioabsorbable material. In some embodiments, the occluder is formed from a tube, which is cut to produce struts in each side. Upon the application of force, the struts deform into loops. The radiopaque, bioabsorbable material is a blend of a biocompatible radiopaque material with a bioabsorbable material. In some embodiments, the radiopaque material may have a mass attenuation coefficient greater than about 1.2 cm²/gm and/or a linear attenuation coefficient greater than about 9 cm⁻¹. In some embodiments, the radiopaque material is tungsten. In some embodiments, the bioabsorbable material may have a molecular weight greater than about 300,000. In some embodiments, the bioabsorbable material is a polymer.

### REFERENCE TO RELATED APPLICATIONS

This application claims the benefit of priority to U.S. Provisional Patent Application Ser. No. 60/729,549, filed Oct. 24, 2005, the disclosure of which is incorporated by reference herein in its entirety.

### FIELD OF THE INVENTION

The present invention relates generally to an occlusion device for the closure of physical anomalies, such as an atrial septal defect, a patent foramen ovale, and other septal and vascular defects. The invention also relates to making such a device or other medical implant radiopaque.

### BACKGROUND OF THE INVENTION

A patent foramen ovale (PFO), illustrated in FIG. 1, is a persistent, one-way, usually flap-like opening in the wall between the right atrium 11 and left atrium 13 of the heart 10. Because left atrial (LA) pressure is normally higher than right atrial (RA) pressure, the flap usually stays closed. Under certain conditions, however, right atrial pressure can exceed left atrial pressure, creating the possibility that blood could pass from the right atrium 11 to the left atrium 13 and blood clots could enter the systemic circulation. It is desirable that this circumstance be eliminated.

The foramen ovale serves a desired purpose when a fetus is gestating in utero. Because blood is oxygenated through the umbilical chord, and not through the developing lungs, the circulatory system of the fetal heart allows the blood to flow through the foramen ovale as a physiologic conduit for right-to-left shunting. After birth, with the establishment of pulmonary circulation, the increased left atrial blood flow and pressure results in functional closure of the foramen ovale. This functional closure is subsequently followed by anatomical closure of the two over-lapping layers of tissue: septum primum 14 and septum secundum 16. However, a PFO has been shown to persist in a number of adults.

The presence of a PFO is generally considered to have no therapeutic consequence in otherwise healthy adults. Paradoxical embolism via a PFO is considered in the diagnosis for patients who have suffered a stroke or transient ischemic attack (TIA) in the presence of a PFO and without another identified cause of ischemic stroke. While there is currently no definitive proof of a cause-effect relationship, many studies have confirmed a strong association between the presence of a PFO and the risk for paradoxical embolism or stroke. In addition, there is significant evidence that patients with a PFO who have had a cerebral vascular event are at increased risk for future, recurrent cerebrovascular events.

Accordingly, patients at such an increased risk are considered for prophylactic medical therapy to reduce the risk of a recurrent embolic event. These patients are commonly treated with oral anticoagulants, which potentially have adverse side effects, such as hemorrhaging, hematoma, and interactions with a variety of other drugs. The use of these drugs can alter a person's recovery and necessitate adjustments in a person's daily living pattern.

In certain cases, such as when anticoagulation is contraindicated, surgery may be necessary or desirable to close a PFO. The surgery would typically include suturing a PFO closed by attaching septum secundum to septum primum. This sutured attachment can be accomplished using either an interrupted or a continuous stitch and is a common way a surgeon shuts a PFO under direct visualization.

Umbrella devices and a variety of other similar mechanical closure devices, developed initially for percutaneous closure of atrial septal defects (ASDs), have been used in some instances to close PFOs. These devices potentially allow patients to avoid the side effects often associated with anticoagulation therapies and the risks of invasive surgery. However, umbrella devices and the like that are designed for ASDs are not optimally suited for use as PFO closure devices.

Currently available septal closure devices present drawbacks, including technically complex implantation procedures. Additionally, there are not insignificant complications due to thrombus, fractures of the components, conduction system disturbances, perforations of heart tissue, and residual leaks. Many devices have high septal profile and include large masses of foreign material, which may lead to unfavorable body adaptation of a device. Given that ASD devices are designed to occlude holes, many lack anatomic conformability to the flap-like anatomy of PFOs. Thus, when inserting an ASD device to close a PFO, the narrow opening and the thin flap may form impediments to proper deployment. Even if an occlusive seal is formed, the device may be deployed in the heart on an angle, leaving some components insecurely seated against the septum and, thereby, risking thrombus formation due to hemodynamic disturbances. Finally, some septal closure devices are complex to manufacture, which may result in inconsistent product performance.

A septal defect closure device can promote tissue growth and healing of the defect. A permanent implant may not be necessary. Bioabsorbable materials can be useful material for implantable devices such as a septal closure device because they degrade over time into non-toxic materials and are absorbed into bodily tissue. The body, therefore, may accept the implant without long-term medication to suppress an immunal or inflammatory response. Bodily tissue may even grow “through” the bioabsorbable material. In addition, because these materials degrade over a known period of time, determined in part by the characteristics of the material, eventually the device will be entirely absorbed by the body. Because the device is absorbed in the body, removal procedures via catheter or invasive surgery are unnecessary.

When devices are implanted percutaneously (e.g., via a catheter) it is important to be able to observe the location and position of the devices by some technique such as fluoroscopy or X-ray. Bioabsorbable materials are typically radiotranslucent and cannot be viewed easily using fluoroscopy or X-ray. This characteristic makes the implantation of a device made of a bioabsorbable material challenging because the position of the device cannot be determined with precision. Some techniques are known for making a bioabsorbable implant partially radiopaque (i.e., can be seen under fluoroscopy or X-ray) so that the position of the device can be viewed during implantation by fluoroscopy and X-ray.

One technique for making a device viewable under fluoroscopy involves attaching a small radiopaque marker “band” at a predetermined location on the device. When marker bands are applied to a device, the location and orientation of the device can be inferred based on the location and orientation of the visible radiopaque marker bands. Accordingly, the device can be delivered under the guidance of fluoroscopy and X-ray equipment to monitor the position of the device relative to the desired implantation site in a patient's body, and to ensure their proper orientation, position, and/or deployment. A radiopaque marker band is usually made of metal because many metals have radiopaque properties.

Devices with radiopaque marker “band(s)” can also suffer from limited visibility. Specifically, the radiographic visibility of a device incorporating a marker band(s) is limited to specified regions of the marker band itself. As noted above, when a marker band is placed on a device, the location of the device at the delivery site is only known by inference relative to the marker band, not by actually viewing the device. Under these conditions, the placement of a device in the body requires alignment of the device based on limited viewing of radiopaque areas.

The use of (non-bioabsorbable) metal radiopaque marker band(s) with bioabsorbable devices may have other potential problems. After the bioabsorbable portion of the device is absorbed, the marker band remains behind and is usually embedded in the surrounding tissues leaving a foreign mass in the body. In addition, the body might recognize a metallic mass as a foreign material, and respond with an immune reaction or long-term inflammation. Such responses can adversely impact the usefulness of a medical implant.

Instead of, or in addition to, a marker band, a radiopaque agent, such as barium sulfate, can be mixed with a non-radiopaque material, to form a radiopaque blend. This agent usually provides a non-radiopaque material with some degree of radiopacity to allow the device made of such radiopaque blend to be visible under fluoroscopy. Generally, the more radiopaque agent that is added in the blend, the greater radiopacity that can be achieved.

Adding a radiopaque agent to bioabsorbable material presents design considerations and challenges. One such consideration is whether the radiopaque agent can be safely processed, e.g. degraded, absorbed, and/or excreted, by the body as opposed to generating an inflammatory reaction, toxicin, or being collected in an organ such as the liver.

Additionally, even if a radiopaque agent can be safely processed by the body, the addition of the radiopaque agent to the non-radiopaque material changes the mechanical and/or thermal properties of the non-radiopaque material. The viscosity, maximum stress, modulus, elongation, and glass transition temperature of the non-radiopaque material may all be significantly altered by the presence of the radiopaque agent.

Therefore, there is a need for an improved radiopaque septal defect closure device that can be viewed through fluoroscopy and X-ray and can also be safely processed by the body.

The present invention is designed to address these and other deficiencies of prior art septal closure devices.

### SUMMARY OF THE INVENTION

In one aspect, the present invention provides an occluder for a biological defect to be introduced into the body through the vasculature. In one aspect of the invention, the occluder includes a structural member formed of a radiopaque bioabsorbable material which is made of a blend of a bioabsorbable material and a radiopaque material. In certain embodiments, the radiopaque bioabsorbable material has a thickness between 500 and 750 microns. In certain embodiments, the radiopaque material has a linear attenuation coefficient greater than about 9 cm⁻¹, and the radiopaque bioabsorbable material contains between about 20 to about 35 percent by weight of the radiopaque material, and preferably between 20 to 35 percent by weight of the radiopaque material.

In certain embodiments, the bioabsorbable material is selected from a group consisting of polyglycolic acid, polylactic acid, poly caprolactone, poly (hyrodxybutyrate), poly(hydroxyvalerate), poly(sebacic acid-headecanoic acid anhydride), polyorthoester, polydioxanone, polygluconate, poly(amino acid), poly(alpha hydroxyl acid), and co-polymers thereof. In certain embodiments, the radiopaque material is tungsten in the form of a powder. In certain embodiments, the tungsten powder has a particle size in the range between about 0.5 to about 2.0 microns.

In another aspect of the invention, a structural member of an occluder for a biological defect is made of a radiopaque, bioabsorbable material. The material is preferably a blend of a bioabsorbable polymer having a molecular weight of 300,000 or greater and a radiopaque agent. According to some embodiments, the radiopaque agent preferably has a linear attenuation coefficient greater than about 9 cm⁻¹. According to some embodiments, the radiopaque material has a mass attenuation coefficient greater than about 1.2 cm²/gm

According to at least some embodiments, the device is formed from a tube. According to some embodiments, the occluder has a proximal side and a distal side that cooperate to close the defect, and at least one of the proximal side or the distal side includes petals that are formed by the structural member made of a bioabsorbable radiopaque material. According to some embodiments, the occluder further includes tissue scaffolding attached to the occluder.

According to some embodiments, the bioabsorbable material is a bioabsorbable polymer. According to some embodiments, the bioabsorbable polymer is selected from a group consisting of polyglycolic acid, polylactic acid, poly caprolactone, poly (hyrodxybutyrate), poly(hydroxyvalerate), poly(sebacic acid-headecanoic acid anhydride), polyorthoester, polydioxanone, polygluconate, poly(amino acid), poly(alpha hydroxyl acid), and co-polymers thereof.

According to some embodiments, the radiopaque material is tungsten in the form of a powder. According to some embodiments, the tungsten powder has a particle size in the range between about 0.5 and about 2.0 microns and preferably is between 0.5 and 2.0 microns. According to some embodiments, the weight percent of tungsten in the radiopaque bioabsorbable material is in the range between about 20 and 35 weight percent.

According to some embodiments, the radiopaque, bioabsorbable material has a thickness between 500 and 750 microns.

According to some embodiments, the occluder is made from a tube with slits that form petals when the tube changes from a delivery configuration to a deployed configuration. According to some embodiments, the occluder has a proximal side and a distal side that cooperate to close the defect and the proximal side includes proximal petals and the distal side includes distal petals. According to some embodiments, the occluder further comprises tissue scaffolding attached to at least one of the distal petals or the proximal petals. In certain embodiments, the occluder is a patent foramen ovale (PFO) occluder. In some embodiments, the tube consists essentially of the radiopaque bioabsorbable material.

According to some embodiments, the bioabsorbable material is a bioabsorbable polymer. According to some embodiments, the bioabsorbable polymer is selected from a group consisting of polyglycolic acid, polylactic acid, poly caprolactone, poly (hyrodxybutyrate), poly(hydroxyvalerate), poly(sebacic acid-headecanoic acid anhydride), polyorthoester, polydioxanone, polygluconate, poly(amino acid), poly(alpha hydroxyl acid), and co-polymers thereof.

According to some embodiments, the radiopaque material is tungsten in the form of a powder. According to some embodiments, the tungsten powder has a particle size in the range between about 0.5 and about 2.0 microns and preferably is between 0.5 and 2.0 microns. According to some embodiments, the weight percent of tungsten in the radiopaque bioabsorbable material is in the range between about 20 and 35 weight percent.

According to some embodiments, the radiopaque, bioabsorbable material has a thickness between 500 and 750 microns.

In another aspect of the invention, includes implanting a radiopaque, bioabsorbable occluder is implanted by insertion into the vasculature of a body. One aspect of the invention includes a method of implanting an occluder for a biological defect, including the steps of providing an occluder, having a structural member consisting essentially of a radiopaque bioabsorbable material. The radiopaque bioabsorbable material comprises a blend of a bioabsorbable material and a radiopaque material. The bioabsorbable material has a molecular weight of at least about 300,000, and the radiopaque material having a linear attenuation coefficient greater than about 9 cm⁻¹. The occluder is inserted into a subject using a catheter and the position and orientation of the device are viewed radiographically during implantation.

In another aspect of the invention, a method of making a radiopaque, bioabsorbable medical implant is provided. A method of making a radiopaque, bioabsorbable medical implant having a structural member formed of a blended radiopaque bioabsorbable material, made of a bioabsorbable polymer and a radiopaque agent, includes the following steps. A biocompatible radiopaque agent for blending with a bioabsorbable polymer is selected. A concentration of the radiopaque agent in the radiopaque bioabsorbable material to attain a desired level of radiopacity is determined. For a physical property of the radiopaque bioabsorbable material that will vary as the material is bioabsorbed after implantation, a desired initial criteria is identified and a bioabsorbable polymer is selected according to the desired initial criteria. The selected radiopaque agent and the selected bioabsorbable polymer are blended according to the determined concentration to form the blended material and the structural member is formed using the blended material. According to certain embodiments, the desired initial criteria is determined based on an expected rate of bioabsorption and an expected life of the implant. According to certain embodiments, the physical property is molecular weight. According to certain embodiments, the radiopaque agent is tungsten. According to certain embodiments, the concentration of the radiopaque agent is between about 20 and 35 weight percent. According to certain embodiments, the bioabsorbable polymer is selected from a group consisting of polyglycolic acid, polylactic acid, poly caprolactone, poly (hyrodxybutyrate), poly(hydroxyvalerate), poly(sebacic acid-headecanoic acid anhydride), polyorthoester, polydioxanone, polygluconate, poly(amino acid), poly(alpha hydroxyl acid), and co-polymers thereof.

### BRIEF DESCRIPTION OF THE DRAWINGS

FIG. 1 is a schematic representation of a human heart including various septal defects;

FIGS. 2A-2D are isometric views of an embodiment of an occluder according to the present invention;

FIGS. 2E-2H are isometric views of an embodiment of an occluder according to the present invention;

FIGS. 2I-2K are isometric views of occluders according to various embodiments of the invention;

FIGS. 2L and 2M are side and top views, respectively, of an alternate embodiment of an occluder according to the present invention;

FIGS. 3A-3C are front elevational, side, and cross-sectional views, respectively, of the occluder of FIGS. 2A-2D;

FIGS. 4A-4B are front elevational and side views, respectively, of another embodiment of an occluder according to the present invention;

FIGS. 5A-5B are front and side views, respectively, of still another embodiment of an occluder according to the present invention;

FIGS. 6A-6E are isometric views of one embodiment of a catch system according to the present invention;

FIGS. 7A-7C are side views of another embodiment of a locking mechanism according to the present invention;

FIGS. 8A-8C are isometric views of yet another embodiment of an occluder according to the present invention;

FIGS. 9A-9H are side views of one method for delivering an occluder according to the present invention to a septal defect; and

FIGS. 10A-10D are side views of one method for retrieving an occluder according to the present invention from a septal defect;

FIG. 11 is a side view of an embodiment of the occluder of the present invention;

FIG. 12 is an isometric view of an embodiment of the occluder of the present invention; and

FIG. 13 is a side view of the occluder of FIGS. 21-2K deployed in vivo.

### DETAILED DESCRIPTION OF THE INVENTION

The present invention provides a device for occluding an aperture within body tissue. This device relates particularly to, but is not limited to, a septal occluder made from a polymer tube. In particular and as described in detail below, the occluder of the present invention may be used for closing an ASD or PFO in the atrial septum of a heart. Although the embodiments of the invention are described with reference to an ASD or PFO, one skilled in the art will recognize that the device and methods of the present invention may be used to treat other anatomical conditions. As such, the invention should not be considered limited in applicability to any particular anatomical condition.

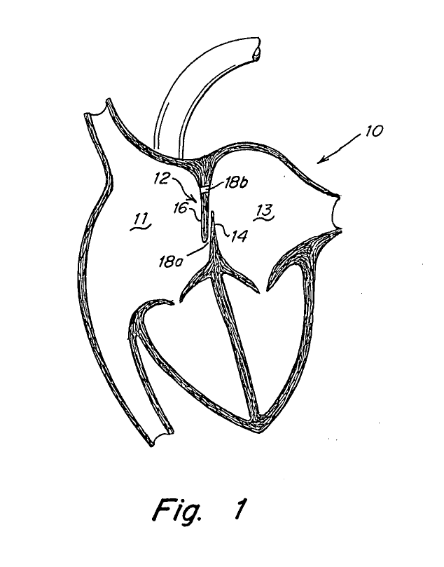

FIG. 1 illustrates a human heart 10, having a right atrium 11 and a left atrium 13 and including various anatomical anomalies 18a and 18b. The atrial septum 12 includes septum primum 14 and septum secundum 16. The anatomy of the septum 12 varies widely within the population. In some people, septum primum 14 extends to and overlaps with septum secundum 16. The septum primum 14 may be quite thin. When a PFO is present, blood could travel through the passage 18a between septum primum 14 and septum secundum 16 (referred to as “the PFO tunnel”). Additionally or alternatively, the presence of an ASD could permit blood to travel through an aperture in the septum, such as that schematically illustrated by aperture 18b.

The term “bioabsorbable,” as used in this application, is also understood to mean “bioresorbable.”

In this application, “distal” refers to the direction away from a catheter insertion location and “proximal” refers to the direction nearer the insertion location.

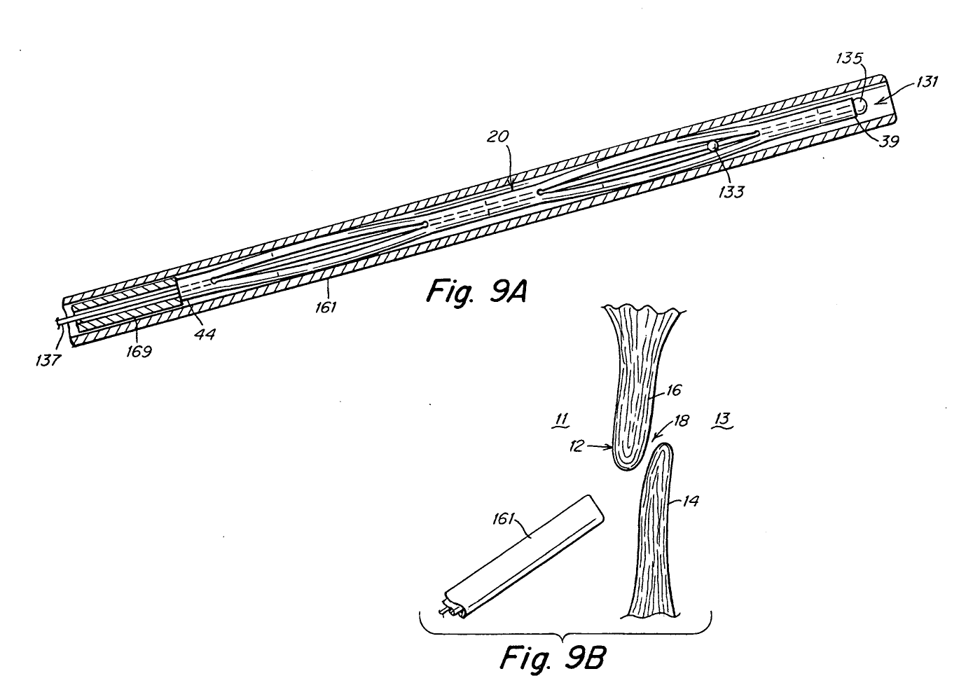

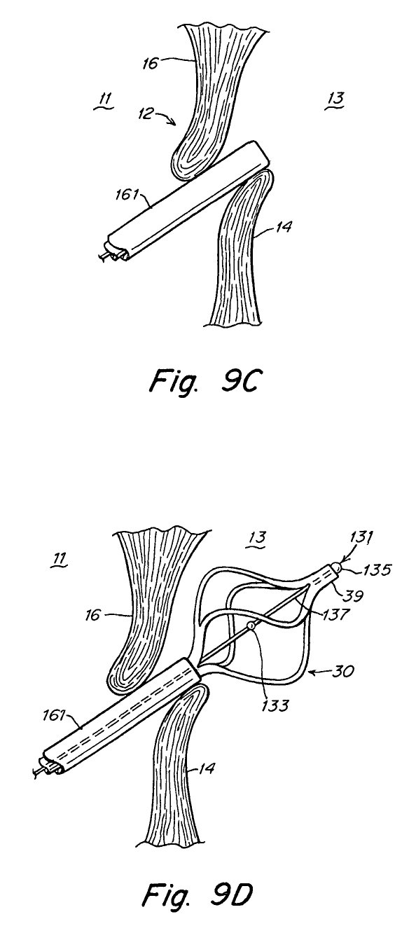

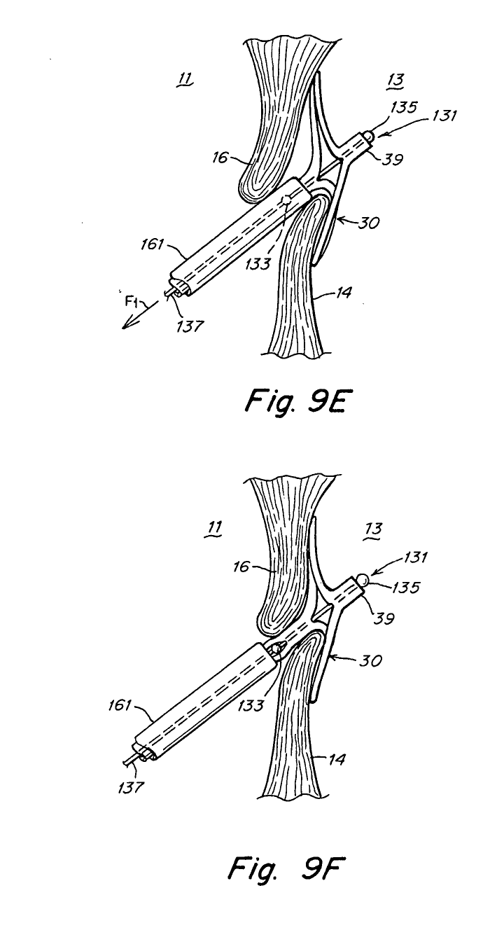

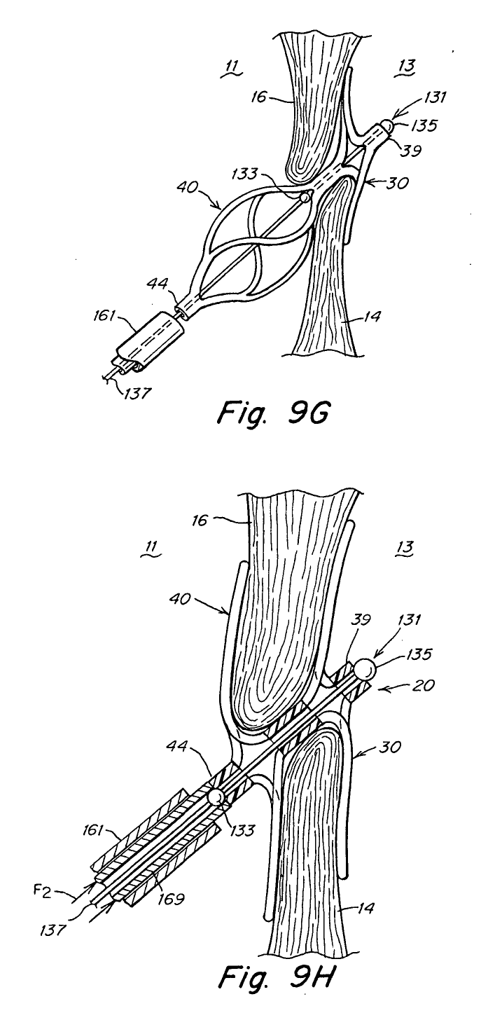

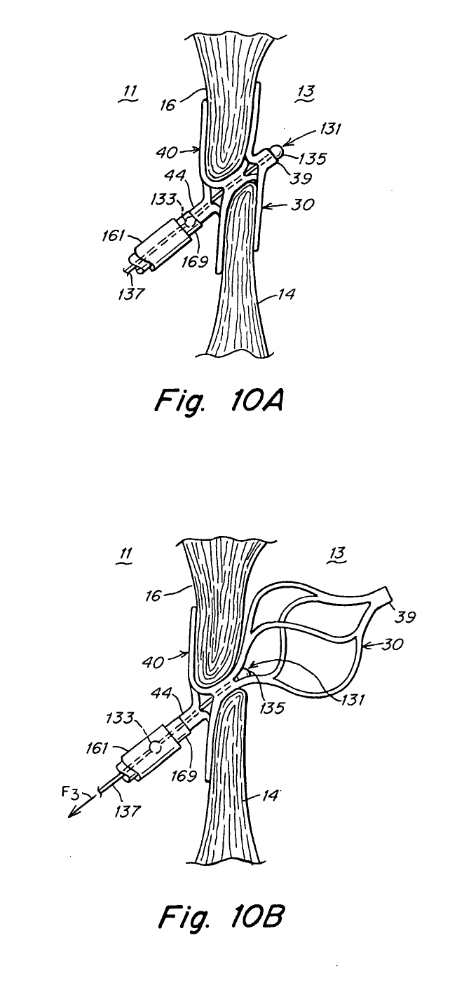

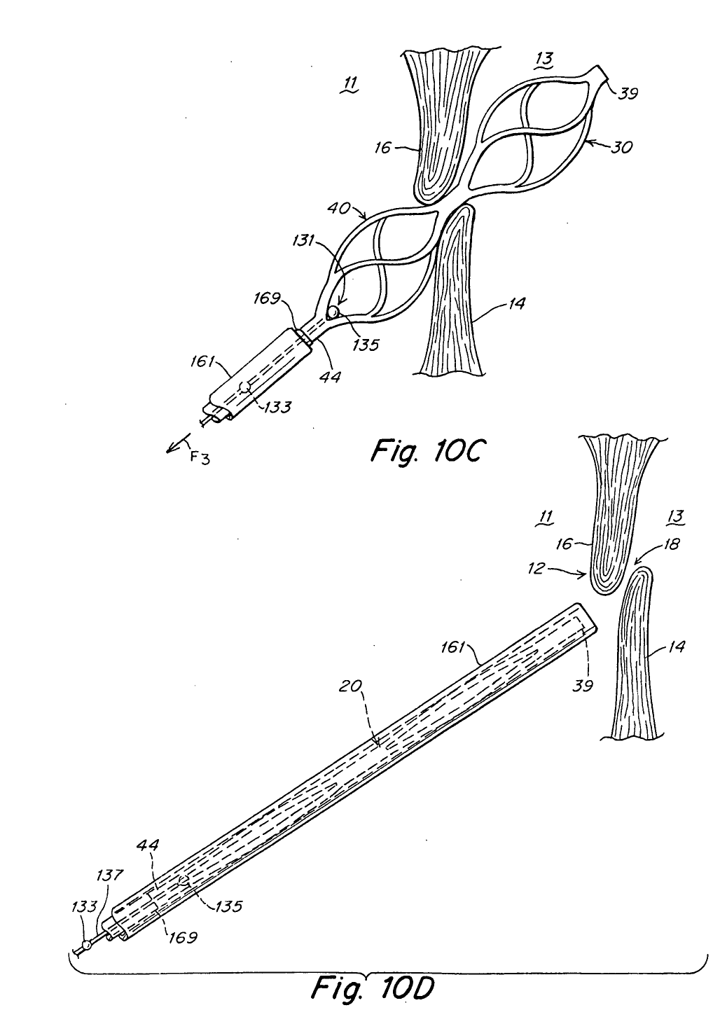

Referring to occluder 20, distal side 30 and proximal side 40 are connected by central tube 22. As illustrated, e.g., in FIGS. 9 and 10 the central tube 22 is an uncut central part of the tube used to form occluder 20. As described below, the entire tube is indicated by reference numeral 25. As shown in FIGS. 9 and 10, the occluder 20 may be inserted into the septum 12 to prevent the flow of blood through the aperture 18a, e.g., the occluder may extend through the PFO tunnel such that the distal side 30 is located in the left atrium 13 and the proximal side 40 is located in the right atrium 11. Additionally or alternatively, the occluder 20 may be inserted into the septum 12 so as to prevent the flow of blood through the aperture 18b, e.g., the occluder may extend through the ASD such that the distal side 30 is located in the left atrium 13 and the proximal side 40 is located in the right atrium 11. As used in this application, unless otherwise indicated, the term “aperture 18” refers to any anatomical anomaly that may be treated by use of occluder 20, such as PFO 18a or ASD 18b.

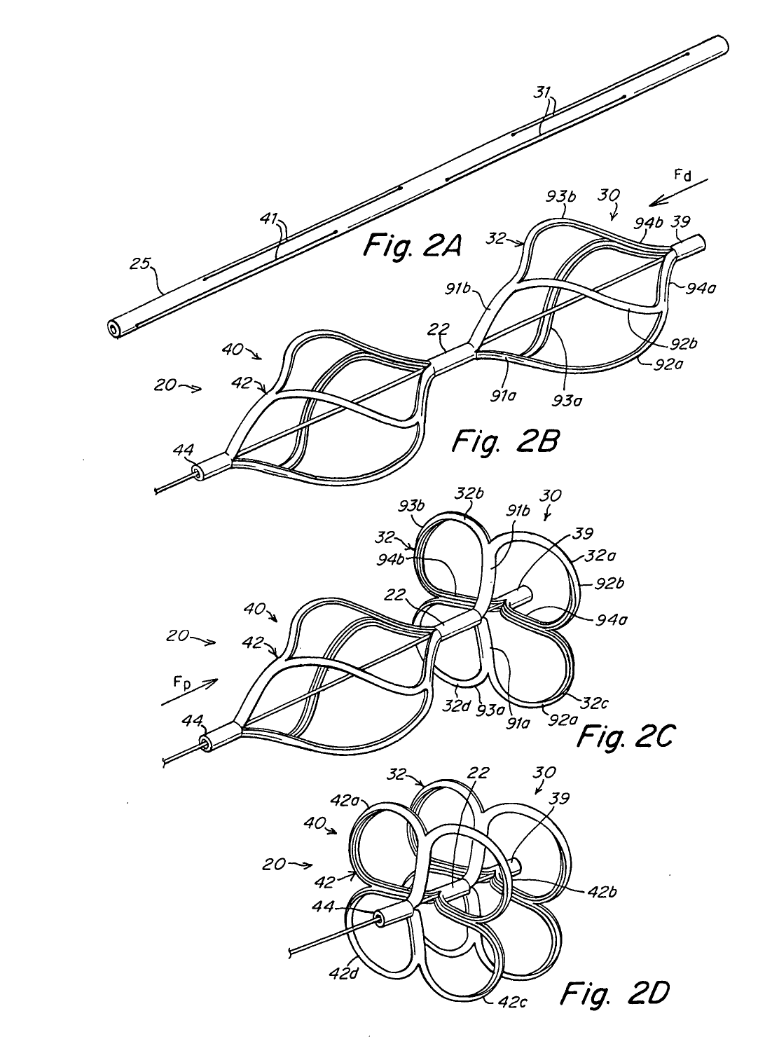

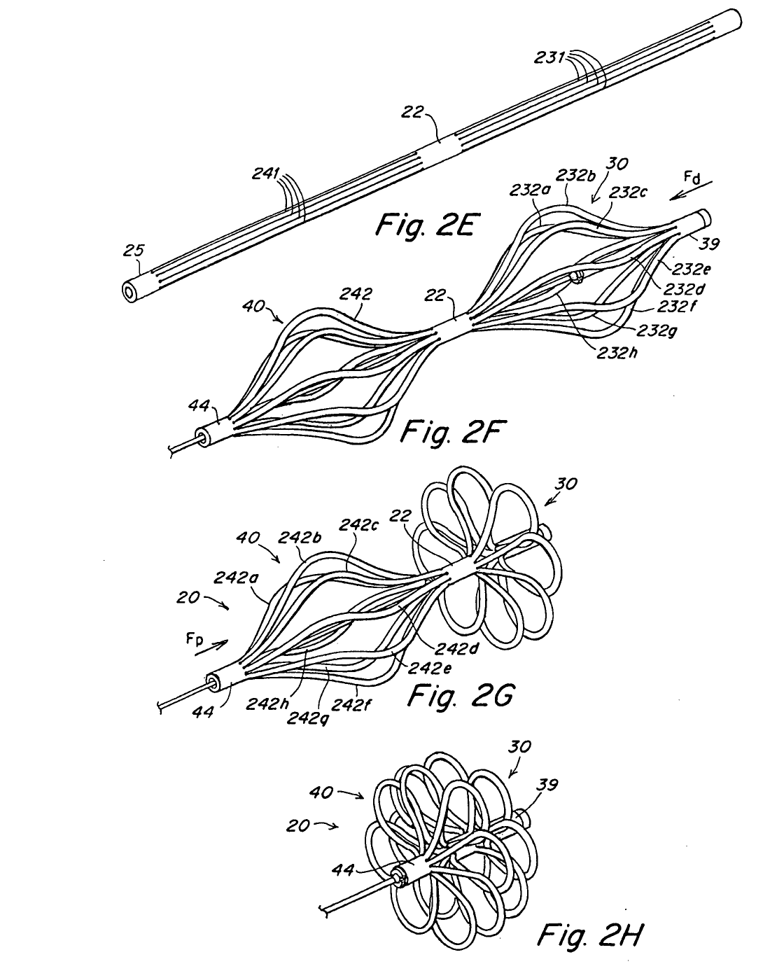

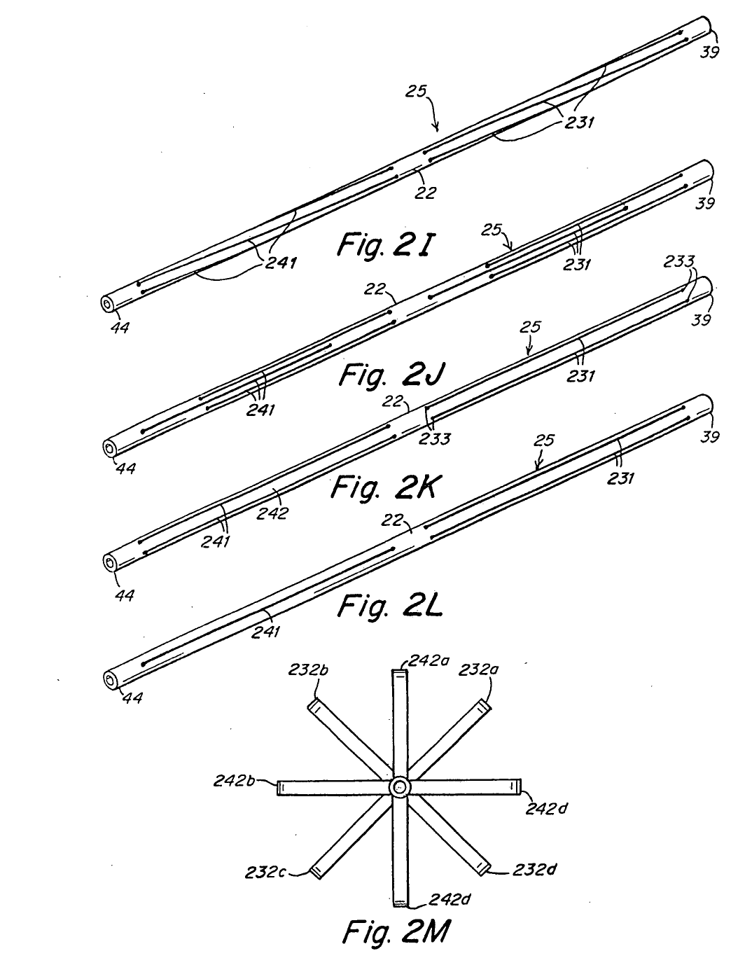

The occluder 20 is constructed of one or more metal or polymer tube(s), referred to collectively as “tube” 25. Tube 25 includes slits 31 and 41 (or 231 and 241), which are formed using an etching or cutting process that produces a particular cutting pattern on tube 25. For example, as shown in FIG. 2K, slits 31 (or 231) are cut along the axial length of the upper half of tube 25 using a cutting tool, e.g., a razor blade. According to some embodiments of the present invention and as shown in FIG. 2K, slits 31 (or 231) are cut without removing any significant amount of material from tube 25, i.e., the formation of slits 31 (or 231) does not significantly reduce the overall volume of tube 25. According to other embodiments of the present invention, slits 31 (or 231) are formed by cutting material out of tube 25 such that the volume of tube 25 is reduced. Both ends of each of slits 31 are rounded so as to relieve stresses at the axial ends of the slits 31. This prevents slits 31 from lengthening due to cyclic stresses present in a beating heart and the resultant material fatigue. In those embodiments where slits 31 are cut without removing any significant amount of material from tube 25, rounded ends or holes 33 may be produced by burning holes at both ends of each of slits 31. In those embodiments where slits 31 are formed by cutting material out of tube 25, rounded ends 33 may be formed during the cutting process. The size of rounded ends 33 may vary depending upon the dimensions of tube 25 and the amount of stress release required by the deformation.

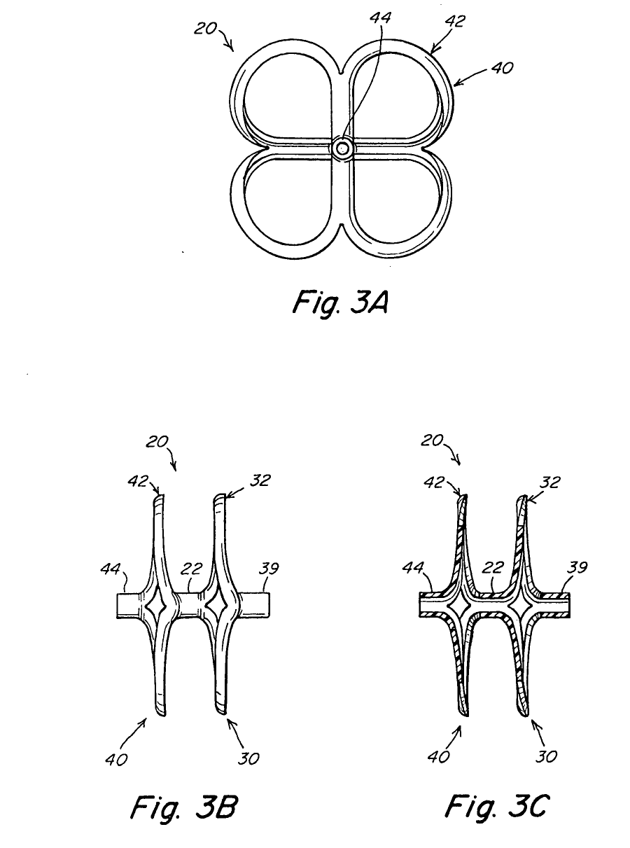

FIG. 2D and 2H illustrate exemplary occluder 20 formed from a tube 25, according to some embodiments of the present invention. Configuration of the occluder 20 is determined by the cutting pattern on tube 25. For example, and as shown in FIGS. 2A, 2B-2D, and 3A-3C, petal-shaped loops 32, 42 (FIGS. 2A-2D and FIG. 3A) are produced by cutting slits 31 in the distal side 30 of tube 25, and cutting slits 41 in the proximal side 40 of tube 25 according to the cutting pattern shown in FIG. 2A. As shown in FIG. 2B, the distal side 30 of tube 25 is cut in half from a center portion 22 to a distal distance to form half sections 91a and 91b. The half sections 91a and 91b are further cut to a proximal distance from the distal end 39 into quarter sections 92a, 93a, 92b, and 93b. The cuts are discontinued and quarter sections 92a and 92b form half section 94a at end 39, and quarter sections 93a and 93b form half section 94b at end 39. Upon application of force F_{d} to end 39, struts bow and twist outward to form petal-shaped loops 32 in distal side 30, as shown in FIGS. 2C-2D. The movement of the struts during deployment is such that the struts rotate in an orthogonal plane relative to the axis of the device. Central tube 22 may be constrained during the application of force F_{d}, or any combination of forces sufficient to reduce the axial length of the tube 25 may be applied. One end of each of petal-shaped loops 32 originates from central tube 22, while the other end originates from end 39 (FIGS. 2B-2C and FIG. 3A). Petal-shaped loops 42 may be formed in proximal side 40 of tube 25, as shown in FIGS. 2B-2D, using the same cutting pattern described above.

Given that the surface of occluder 20 will contact septum 12 once it is deployed in vivo, slits 31 and 41 are cut so as to prevent the formation of sharp, potentially damaging edges along their length. For example, a heated cutting tool may be used to cut slits 31 and 41 such that the material of tube 25 melts slightly when placed in contact with the cutting tool. Such melting rounds the edges of the sections. Lasers may also be used to cut slits 31 and 41. According to this process, the edges of loops 32 and 42 formed by the cutting of slits 31 and 41 are blunted (due to melting) to prevent tissue damage in vivo. One skilled in the art will recognize that same considerations and techniques also apply to slits 231 and 241.

The tube(s) 25 forming occluder 20 includes a biocompatible metal or polymer. In at least some embodiments, the occluder 20 is formed of a bioabsorbable polymer, or a shape memory polymer. In other embodiments, the occluder 20 is formed of a biocompatible metal, such as a shape memory alloy (e.g., nitinol). The thermal shape memory and/or superelastic properties of shape memory polymers and alloys permit the occluder 20 to resume and maintain its intended shape in vivo despite being distorted during the delivery process. In addition, shape memory polymers and metals can be advantageous so that the structure of the device assists in compressing the PFO tunnel closed. Alternatively, or additionally, the occluder 20 may be formed of a bioabsorbable metal, such as iron, magnesium, or combinations of these and similar materials. Exemplary bioabsorbable polymers include polyhydroxyalkanoate compositions, for example poly-4-hydroxybutyrate (P4HB) compositions, disclosed in U.S. Pat. No. 6,610,764, entitled Polyhydroxyalkanoate Compositions Having Controlled Degradation Rate and U.S. Pat. No. 6,548,569, entitled Medical Devices and Applications of Polyhydroxyalkanoate Polymers, both of which are incorporated herein by reference in their entirety.

In certain embodiments, the occluder 20 is partially or completely radiopaque, and, in particular, is partially or completely formed of radiopaque bioabsorbable materials.

Preferred materials for making the occluder 20 are radiopaque under standard X-ray and fluoroscopy equipment and bioprocessable, for example through absorption, degradation, excretion, or otherwise processed (generally referred to as bioabsorbed) safely by the body over a predetermined period of time. In addition, in preferred embodiments, the implantation and subsequent absorption of the material will not cause safety concerns, including inflammation, toxicity, tissue accumulation and rejection. As the material is absorbed, the device “disappears” so as not to leave any implant behind, and the radiopaque agent does not create an embolic risk but degrades and is excreted from the body.

The radiopaque bioabsorbable materials can be used as a main component, e.g. a structural member, or a frame, of a medical implant such as occluder frame 20, allowing all or nearly all of the implant to be monitored with radiography during implantation.

Further, by selecting an appropriate bioabsorbable material, the device can be manufactured to be bioabsorbed over a desired period of time, and/or with proper in-growth of native tissue. For example, in the case of an occlusive device designed to seal a hole or a defect in tissue such as occluder 20, it might be desirable that the bioabsorption process is not completed until the hole completely heals through regeneration of native tissue. For example, native tissue growth may begin in 6 months after implantation and complete healing may take two years. The radiopaque characteristics of the materials of preferred embodiments will allow the progress kinetics of the bioabsorption of the device, as well as any changes in the device location and orientation, to be easily monitored.

According to preferred embodiments, a radiopaque, bioabsorbable occluder 20 is formed from a bioabsorbable material selected as a base material, e.g., a bioabsorbable polymer, preferably blended with a radiopaque agent to form a radiopaque bioabsorbable material. In a preferred embodiment, the radiopaque agent is biocompatible and is capable of being broken down in the body and flushed out so that the radiopaque agent does not accumulate in major organs, such as the liver.

Additionally, the radiopaque agent at a suitable concentration to obtain the desired physical properties of the blended material should provide sufficient radiopacity so that during fluoroscopic examination the material can be reliably viewed. The choice of a radiopaque agent

on many factors including the biocompatibility of the agent, the bioabsorbability of the agent and the impact of the radiopaque agent on the structural integrity of a device constructed with the radiopaque bioabsorbable material. One important consideration is the ability of the material to “attenuate” the mono energetic photons emitted by the fluoroscopic and X-ray device, this quality is described as mass attenuation coefficient, (μ/ρ), where μ is linear attenuation coefficient, and ρ is the density of the material. The mass attenuation coefficient of a material is directly related to the visibility of the material under fluoroscopy and X-ray, i.e., its radiopacity.

The relationship can be expressed thus:
I/I₀=exp[−(μ/ρ)χ] (1)
Where a narrow beam of mono energetic photons with an incident intensity of I₀, penetrates a layer of material with mass thickness, χ, and density, ρ, and emerges with an intensity, I. The equation can be rewritten as:
μ/ρ=χ⁻¹ln(I₀/I) (2)
mass thickness, χ, is defined at the mass per unit area and is obtained by multiplying the thickness t by the density, ρ, i.e. χ=ρ·t. The equation can be further rewritten as
−(μ/ρ)χ=−(μ/ρ)(ρ·t)=−(μ·t) (3)
I/I₀=exp[−(μ/ρ)χ]=exp[−(μ·t)] (4)

Accordingly, assuming the same thickness t, the degree of radiopacity is largely determined by the material's linear attenuation coefficient, μ, since a higher linear attenuation coefficient, μ, will result in a lower emerging intensity, I, which indicates an higher degree of radiopacity. Furthermore, to obtain the same degree of radiopacity, i.e. the same emerging intensity, I, a lower thickness, t, of the material with a higher linear attenuation coefficient, μ, will meet the requirement. Considering the linear attenuation coefficient, μ, in the table below, a preferred material for blending with a bioabsorbable material will have a linear attenuation coefficient larger than 35 cm⁻¹. Of course, in other embodiments of the invention, materials with smaller linear attenuation coefficient, μ, could be used with corresponding decreases in their radiopacity.

The table below provides several elements that can be used as the radiopaque agent. In one embodiment of the invention biocompatible materials with mass attenuation coefficient, (μ/ρ), of 1.20 cm²/g or greater are suitable for various embodiments of the invention. In another embodiment, materials with greater density, ρ, and greater mass attenuation coefficient, (μ/ρ), are preferred.

TABLE 1

| | | | Mass | | |
|---|---|---|---|---|---|
| | | | Attenuation | | |
| | Chemi- | | Coefficient | | Linear |
| Atomic | cal | | @60 KeV X- | Density | Attenuation |
| Number | Symbol | Element | ray energy | (g/cm³) | Coefficient |
| 26 | Fe | Iron | 1.21 cm²/g | 7.87 | 9.523 cm⁻¹ |
| 28 | Ni | Nickel | 1.51 cm²/g | 8.9 | 13.439 cm⁻¹ |
| 30 | Zn | Zinc | 1.76 cm²/g | 7.13 | 12.549 cm⁻¹ |
| 34 | Se | Selenium | 2.34 cm²/g | 4.8 | 11.232 cm⁻¹ |
| 42 | Mo | Molyb- | 4.27 cm²/g | 10.2 | 43.554 cm⁻¹ |
| | | denum | | | |
| 53 | I | Iodine | 7.58 cm²/g | 4.94 | 37.445 cm⁻¹ |
| 56 | Ba | Barium | 8.51 cm²/g | 3.6 | 30.636 cm⁻¹ |
| 74 | W | Tungsten | 3.71 cm²/g | 19.3 | 71.603 cm⁻¹ |
| 83 | Bi | Bismuth | 5.233 cm²/g | 9.8 | 51.283 cm⁻¹ |

A preferred radiopaque agent has a mass attenuation coefficient, (μ/ρ), greater than 1.2 cm²/g, more preferably above 3.0 cm²/g. A preferred radiopaque agent has a linear attenuation coefficient, μ, greater than 9 cm⁻¹, more preferably above 30 cm⁻¹. For a material with greater radiopacity, only a small amount would provide a sufficient degree of radiopacity for the resulting material, and therefore, properties of the original material would not be significantly adversely affected by the presence of the radiopaque agent. Specifically, when blending such radiopaque agent with a bioabsorbable material, the mechanical and/or thermal properties of the bioabsorbable material are maintained within an appropriate range.

A particularly preferred radiopaque agent for some embodiments of the invention is tungsten. Tungsten has a linear attenuation coefficient, μ, of 71.603 cm⁻¹, a mass attenuation coefficient, (μ/ρ), of 3.71 cm²/g, and is denser than some other materials that could be considered for use as radiopaque agents, such as barium sulfate. Therefore a much smaller amount of tungsten than barium sulfate would provide an equivalent level of radiopacity in the resulting blend. Because a relatively small amount of tungsten is needed, the mechanical and/or thermal properties of the blend relative to the base polymer should have less degradation than otherwise. For example, in one or more embodiments, only 20-35 weight percent, or 7.6-9.6 volume percent, tungsten in the blend provides a useful level of radiopacity. Additionally, because the resulting blend is highly radiopaque, implantable devices can be fabricated with relatively thin features, while still being easily visualized with standard radiographic equipment.

In addition to being highly radiopaque, tungsten is also biocompatible. As the bioabsorbable material degrades and its byproducts of that process are absorbed or excreted by the body. A similar process occurs for the tungsten. Tungsten has been commonly used in embolization coils. It is known that the coils degrade and disappear over time in the body. As tungsten degrades, it can be eliminated readily from the body, primarily in the urine. In general, the presence of elevated tungsten levels in the blood does not appear to have a detrimental affect on human health.

The degradation of the radiopaque agent, including tungsten could be controlled by selecting the amount of tungsten and the particle size used. The particle size of radiopaque agents identified above, including tungsten, preferably ranges from 0.5 microns to 400 microns. The smaller the particle size, the less property degradation of the original bioabsorbable material. The volume of the radiopaque agents, identified above, including tungsten that can be added to the blend could range from 1% to 80%.

The bioabsorbable material used can be one or more of a variety of bioabsorbable materials known in the art. For example, the bioabsorbable material can be a polymer of glycolide (commonly referred to as polyglycolic acid), a polymer of lactide (commonly referred to as polylactic acid), polycaprolactone, poly(hydroxybutyrate), poly(hydroxyvalerate), poly(sebacic acid-hexadecanoic acid anhydride), polyorthoesters, polydioxanone, polygluconate, poly(amino acids), poly(alpha hydroxy acids), co-polymers of the above (for example poly(galactide-co-lactide) which is commonly referred to as poly glycoic and lactic acid or PGLA), and other bioabsorbable materials, including collagen-based materials. Exemplary bioabsorbable polymers include polyhydroxyalkanoate compositions, for example poly-4-hydroxybutyrate (P4HB) compositions, disclosed in U.S. Pat. No. 6,610,764, entitled Polyhydroxyalkanoate Compositions Having Controlled Degradation Rate and U.S. Pat. No. 6,548,569, entitled Medical Devices and Applications of Polyhydroxyalkanoate Polymers, both of which are incorporated by reference in their entirety. Poly (hydroxybutyrate), in particular, is a preferred material for use as a base material.

The bioabsorption of the radiopaque, bioabsorbable material changes the structure and physical properties and chemical composition of the device over time. It may be medically necessary for the device to function with a certain level of performance for a certain amount of time. In one embodiment, the degradation rate of the radiopaque bioabsorbable material described herein can be controlled by properly selecting the bioabsorbable material, the amount of radiopaque agent blended and the particle size of the radiopaque agent blended. In another embodiment, devices made of the radiopaque bioabsorbable material described in the present invention could have a controlled degradation rate, and desired mechanical and/or thermal properties that meet the requirements of the particular application over time, for example the proper in-growth of native tissue. Selection of a suitable bioabsorbable material, factoring into account the bioabsorption of the blended material, is discussed further.

In one aspect of the invention, the bioabsorbable base material is selected such that the blend will have certain characteristics suitable for a particular application factoring in both the amount of radiopaque agent and also the bioabsorbable and degradable nature of the final material, and useful life of the device. Desired criteria for physical properties (e.g., strength) and bioabsorption characteristics can be used to extrapolate desired properties of the starting material. The desired properties can be used to select a starting material.

To achieve a desired degree of radiopacity, about 20-35 weight percent of tungsten is sufficient in certain embodiments of the invention. When combined, for example, with poly (hydroxybutyrate), the resulting material is sufficiently strong to form a reliable occluder device for implantation. As the bioabsorption process takes place, however, the composition of the occluder 20 changes over time. Different processing rates for the base material (e.g., poly (hydroxybutyrate)) and the radiopaque agent cause the concentration of the radiopaque agent and the strength of the frame 20 to change over time. Using a predicted degradation rate, a desired life span and minimum characteristic of the device 20, it is possible to calculate a desired starting criteria. In preferred embodiments, the base material used to form occluder frame 20, has a molecular weight greater than 300,000 to give the frame sufficient strength over the useful life of the device until the septal defect has healed.

The described radiopaque bioabsorbable material could be made by mixing the selected radiopaque agent with the selected bioabsorbable material together. This could be done by grinding the selected radiopaque agent into fine powder and physically blending the fine radiopaque agent powder with melted or non melted bioabsorbable material to form a composite. This could also be done by melting the selected radiopaque agent and mixing the melt with melted or non melted bioabsorbable material to form a composite. The process of making the composite could take place in an extruder or other machines. The composite could then be used to make implantable devices. The methods of making implantable devices with the composite include, but are not limited to, injection molding, extrusion, thermoforming, casting, and rotational molding.

In one embodiment, the radiopaque bioabsorbable material for occluder 20 can be manufactured by blending fine tungsten particles and the bioabsorbable material together in an extruder. In a preferred embodiment, the tungsten particles can be, for example, between 0.5 and 2.0 microns in diameter. In another preferred embodiment, the weight percent of tungsten in the resulting material is between about 20% and about 35%, preferably between 20% and 35%. In another preferred embodiment, the material is extruded to a thickness of, for example, between 500 and 750 microns to form the occluder. As the bioabsorbable material degrades and its byproducts are absorbed and excreted by the body, tungsten particles are exposed, processed and excreted readily from the body, primarily in the urine.

The cross-sectional shape of tube 25 may be circular or polygonal, for example square, or hexagonal. The slits 31 and 41 (or 231 and 241) may be disposed on the face of the polygon (i.e., the flat part) or on the intersection of the faces.

The tube 25 can be extruded or constructed of a sheet of material and rolled into a tube. The sheet of material could be a single ply sheet or multiple ply. The slits that form the struts could be cut or stamped into the tube prior to rolling the tube to connect the ends to form an enclosed cross section. Various geometrical cross sections are possible including circular, square, hexagonal and octagonal and the joint could be at the vertex or along the flat of a wall if the cross section is of a particular geometry. Various attachment techniques could be used to join the ends of the sheet to form a tube, including welding, heat adhesives, non-heat adhesives and other joining techniques suitable for in-vivo application. One advantage of using a blended radiopaque bioabsorbable material for forming one or more structural members or even the whole occluder frame is that a radiopaque occluder 20 can readily be formed using this processing the radiopaque, bioabsorbable material to form the tube.

The surface of tube 25 may be textured or smooth. An occluder 20 having a rough surface produces an inflammatory response upon contact with septum 12 in vivo, thereby promoting faster tissue ingrowth, healing, and closure of aperture 18a (shown in FIG. 1). Such a rough surface may be produced, for example, by shaving tube 25 to produce whiskers along its surface. For example, central tube 22 may include such whiskers. Additionally or alternatively, the surface of tube 25 may be porous to facilitate cell ingrowth.

The distal side 30 of the occluder 20 (also called the “anchor portion”) is shown in FIGS. 2C and 2D. The distal side 30 includes four loops 32a, 32b, 32c, and 32d (collectively referred to as loops 32). As previously described, each of loops 32a-32d are formed by corresponding cut sections 92b, 93b, 92a, 93a, produced by cutting slits 31. The application of force F_{d} to end 39 of tube 25 brings the axial ends of slits 31 together such that struts bow and twist outwardly to form loops 32 of distal side 30 (FIGS. 2B-2C). Central tube 22 may be constrained during the application of force F_{d}. One skilled in the art will recognize that any combination of forces sufficient to reduce the axial length of the tube 25 would be sufficient to deploy the distal side 30 of occluder 20.

As illustrated, the loops 32 are evenly distributed about central tube 22 and end 39. Thus, when the distal side 30 includes four loops 32 (as shown in FIGS. 2C and 2D), the four slits 31 are spaced 90 degrees radially apart. Similarly, when the distal side 30 includes six loops 32, the six slits 31 are spaced 60 degrees radially apart. The angle between radially equally-spaced spaced is determined by the formula (360/n_{d}), where nd is the total number of loops 32.

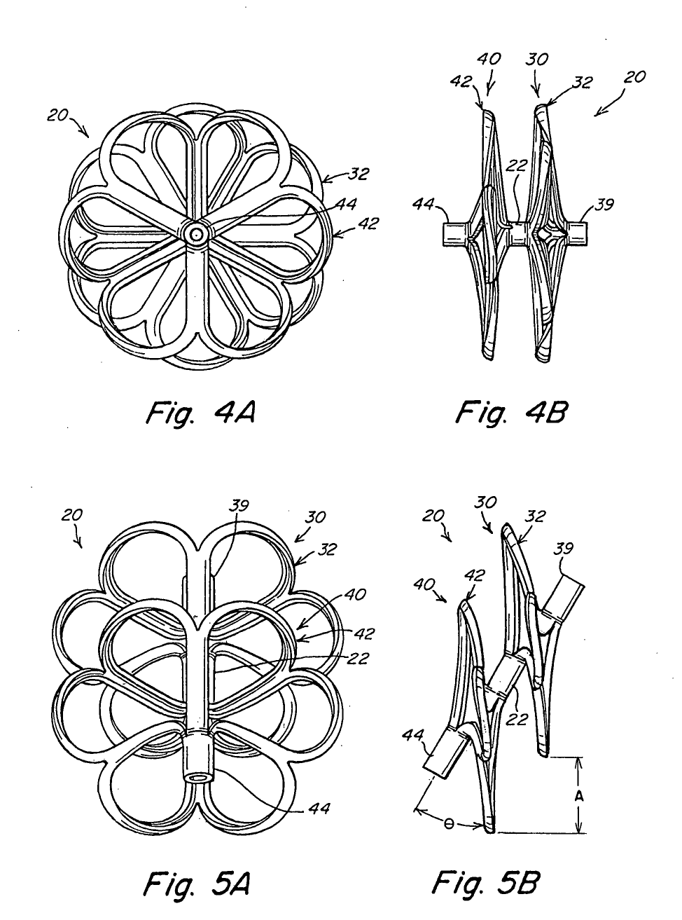

Although the distal side 30 of the occluder 20 shown in FIG. 3A includes four loops 32, occluders according to the present invention may include any number of loops 32 necessary for a given application. In particular embodiments, the distal side 30 of occluder 20 includes six loops 32 (FIG. 4A). Occluders having between four and ten loops 32 may be formed without requiring significant adjustments in the processes described in this application. However, occluders having less than four or more than ten loops 32 may be complicated to manufacture and difficult deliver through the vasculature.

Regardless of the number of loops included in distal side 30 and depending upon the material used to form occluder 20, the outer perimeter of loops 32 may vary. In at least some embodiments, the outer perimeter of loops 32 is rounded to provide an occluder 20 having a smooth, circular perimeter. As the number of loops 32 in the distal side 30 of occluder 20 increases, it becomes desirable to round the outer perimeters of the loops 32 so as to prevent the infliction of trauma on the surrounding septum 12.

The proximal side 40 of the occluder 20, shown in side view in FIG. 2D, also includes four loops, 42a, 42b, 42c, and 42d (collectively referred to as loops 42). As previously described, each of loops 42a-42d are formed by corresponding cut sections, produced by cutting slits 41. The application of force Fₚ to tip 44 of tube 25 brings the axial ends of slits 41 together such that struts bow and twist outwardly to form loops 42 of proximal side 40 (FIGS. 2C-2D). Central tube 22 may be constrained during the application of force Fₚ. One skilled in the art will recognize that any combination of forces sufficient to reduce the axial length of the tube 25 would be sufficient to deploy the proximal side 40 of occluder 20. As described above for distal loops 32, the loops 42 are evenly distributed about central tube 22 and tip 44. Similarly, the angle between radially equally-spaced slits 41 in the proximal side 40 is determined by the formula (360/n_{d}), where nd is the total number of loops 42.

Although the proximal side 40 of the occluder 20 shown in FIG. 2D includes four loops 42, one skilled in the art will recognize that the proximal side 40 of an occluder according to the present invention may include any number of loops 42 required and suitable for a given application. In particular embodiments, the proximal side 40 of occluder 20 includes six loops 42 (FIG. 4A). Further, although as illustrated, distal side 30 and proximal side 40 both include four loops, there is no requirement that distal side 30 and proximal side 40 of occluder 20 include the same number of loops. In fact, in particular applications, it may be advantageous to use an occluder 20 in which the distal side 30 contains fewer loops than the proximal side 40, or vice versa.

It will be apparent to one skilled in the art that loops 32 and loops 42 (or loops 232 and 242) do not have to be the same size. In one embodiment, loops 32 (or 232) are larger in size than loops 42 (or 242). In another embodiment, loops 32 (or 232) are smaller in size than loops 42 (or 242). Size of loops 32 and 42 (or 232 and 242) is determined by the lengths of slits 31 and 41 (or 231 and 241), respectively. Therefore, absolute and relative lengths of slits 31 and 41 (or 232 and 241) can be varied to achieve desired absolute and relative sizes of loops 32 and 42 (or 232 and 242).

In at least some embodiments, illustrated in FIGS. 4A, loops 42 of the proximal side 40 are radially offset from loops 32 of the distal side 30 to provide a better distribution of forces around the aperture 18a. This can be achieved by making cuts to create slits 31 and 41 such that they are radially offset relative to each other. The maximum degree of offset will depend on the number of slits. In general, if slits are equally spaced, the maximum possible offset will be one half of the angle between the loops. For example, if distal side 30 (or proximal side 40) contains 4 slits (and therefore 4 loops), loops will be 90 degrees apart (see the formula described above), thereby allowing for maximum degree of offset of one half of 90 degrees (which is 45 degrees) between loops 32 and loops 42. In a preferred form, when distal side 30 (or proximal side 40) contains 4 slits (and therefore 4 loops), loops 42 and loops 32 are offset by 45 degrees. In an alternative embodiment, the degree of offset between loops 32 and 42 ranges from about 30 to about 45 degrees.

FIGS. 2E-2H illustrate another embodiment of the invention, where the occluder 20 is formed from a tube with loops 232 and 242, produced from the cutting pattern shown in FIG. 2E. In one embodiment, the proximal side 40 and the distal side 30 of occluder 20 each include eight loops or petals. As shown in FIG. 2E, the distal portion 30 of the tube 25 includes 8 slits 231 that form 8 extended segments of the tube that form the distal loops or petals 232. As apparent from the figures, the slits extend the entire distance of the distal portion 30 of the tube 25, i.e. between central tube 22 and distal end 39, so that the loops of identical cross-sections are formed. Upon application of force F_{d} to distal end 39, extended segments defined by slits 231 bow and twist outward to form distal petals 232 in distal side 30 of the occluder 20. The movement of the segments during deployment is such that the segments rotate in an orthogonal plane relative to the axis of the device. Central tube 22 may be constrained during the application of force F_{d}, or any combination of forces sufficient to reduce the axial length of the tube may be applied. One end of each of distal petals 232 originates from central tube 22, while the other end originates from distal end 39. Proximal petals 242 may be formed in proximal portion 40, as shown in FIGS. 2E-2H, making slits 241 between central tube 22 and proximal tip 44, using the same cutting pattern described above and applying force Fₚ or combination of forces sufficient to reduce the axial length of the tube by allowing slits 241 to bow and twist outward to form proximal petals 242 in proximal portion 40 of the occluder 20. One end of each of proximal petals 242 originates from central tube 22, while the other end originates from proximal tip 44.

One embodiment of the distal side 30 of the occluder 20 (also called the “anchor portion”) is shown in FIG. 2G and 2H. The distal side 30 includes eight loops 232a, 232b, 232c, 232d, 232e, 323f, 232g, and 232h (collectively referred to as loops 232). As previously described, each of loops 232a-232h is produced by cutting slits 231. The application of force Fd to end 39 of tube 25 brings the axial ends of slits 231 together such that struts bow and/or twist outwardly to form loops 232 of distal side 30 (FIGS. 2F-2G). Central tube 22 may be constrained during the application of force F_{d}. One skilled in the art will recognize that any combination of forces sufficient to reduce the axial length of the tube 25 would be sufficient to deploy the distal side 30 of occluder 20.

As illustrated, the loops 232 are evenly distributed about central tube 22 and end 39. Thus, when proximal side 30 includes eight loops 232 (as shown in FIGS. 2G and 2H), the eight slits 231 are spaced 45 degrees radially apart. The angle between radially equally-spaced slits 231 in distal side 30 is determined by the formula (360/n_{d}) where n_{d} is the total number of loops 232.

The proximal side 40 of the occluder 20, shown in side view in FIG. 2H, also includes eight loops, 242a, 242b, 242c, 242d, 242e, 242f, 242g, and 242h (collectively referred to as loops 242). As previously described, each of loops 242a-242h is produced by cutting slits 241. The application of force Fₚ to tip 44 of tube 25 brings the axial ends of slits 241 together such that struts bow and twist outwardly to form loops 242 of proximal side 40 (FIGS. 2G-2H). Central tube 22 may be constrained during the application of force Fₚ. One skilled in the art will recognize that any combination of forces sufficient to reduce the axial length of the tube 25 would be sufficient to deploy the proximal side 40 of occluder 20. As described above for distal side 30, the loops 242 are evenly distributed about central tube 22 and tip 44. Similarly, the angle between radially equally-spaced slits 241 in proximal side 40 is determined by the formula (360/n_{d}) where n_{d} is the total number of loops 242.

Although the distal side 30 and the proximal side 40 of the occluder 20, shown in FIG. 2H, each include eight loops 232 and 242, respectively, one skilled in the art will recognize that the distal side 30 and proximal side 40 of an occluder 20 according to the present invention may include any number of loops 232 and 242, respectively, required and/suitable for a given application. Further, although as illustrated, distal side 30 and proximal side 40 both include eight loops, there is no requirement that distal side 30 and proximal side 40 include the same number of loops. In fact, in particular applications, it may be advantageous to use an occluder 20 in which distal side 30 contains fewer loops than proximal side 40, or vice versa.

It will be apparent to one skilled in the art that loops 232 and loops 242 do not have to be the same size. In one embodiment, loops 232 are larger in size than loops 242. In another embodiment, loops 232 are smaller in size than loops 242. Size of loops 232 and 242 is determined by the lengths of slits 231 and 241, respectively. Therefore, absolute and relative lengths of slits 231 and 241 can be varied to achieve desired absolute and relative sizes of loops 232 and 242.

While loops 232 and 242, shown in FIGS. 2F-2H are illustrated as aligned, this does not have to be the case. In one embodiment, loops 232 and 242 are radially offset from each other. This can be achieved by making cuts to create slits 231 and 241 such that they are radially offset relative to each other. The maximum degree of offset will depend on the number of slits. In general, if slits are equally spaced, the maximum possible offset will be one half of the angle between the loops. For example, if distal side 30 (or proximal side 40) contains 8 slits (and therefore 8 loops), the loops will be 45 degrees apart (see the formula described above), thereby allowing for maximum degree of offset of one half of 45 degrees, which is 22.5 degrees between loops 232 and loops 242. It is understood, that offset can be in either rotational direction (i.e., clockwise and counterclockwise). Therefore, in this example with 8 slits, an offset of 30 degrees is equivalent to an offset of 7.5 degrees in the opposite direction.

The cutting pattern illustrated in FIG. 2E can be varied, as shown in FIGS. 2I-2K. According to one embodiment of the invention, the number of slits 231 and 241 cut in the tube 25 can be changed according to the desired number of loops 232 and 242 in the occluder 20 when deployed. The cross-sectional dimensions of loops 232 and 242 are determined by the thickness of tube 25 and the distance between adjacent slits 231 and 241. The length of slits 231 and 241 determines the length of loops 232 and 242 and the radial dimensions of the deployed occluder 20. In this manner, the dimensions of loops 232 and 242 can be controlled during production of occluder 20. For example, as more material is removed from tube 25 during the cutting process used to form slits 231 and 241, the thickness of loops 232 and 242 decreases. Moreover, any or all of slits 231 and 241 can be cut such that thickness of loops 232 and 242 varies along their length. In some embodiments, it may be desirable to have wider loops 232 and 242 at the location where the loops join tube 25 to create a sturdier device. Alternatively, it may be desirable to have a wider portion elsewhere along the loops 232 and 242 such that occluder 20 is predisposed to bend into a certain shape and arrangement. For example, the portion of loops 232 and 242 nearer central tube 22 may be thinner than the portion of loops 232 and 242 nearer end 39 and tip 44, respectively, to facilitate bending of the loops 232 and 242.

Slits 231 and 241, as shown in FIG. 2J, are cut axially along the length of tube 25. However, as one of skill in the art will recognize, slits 231 and/or 241 may also be cut along other dimensions of tube 25. For example, as shown in FIG. 2I, slits 231 and 241 may be cut at an angle such that they are helically disposed on tube 25. Angled slits 231 and 241 produce angled loops 232 and 242 during deployment. Further, slits 231 and 241 need not be straight; for example, slits 231 and 241 may be cut as zigzags, S-shaped slits, or C-shaped slits. One skilled in the art will be capable of selecting the angle for the slits 231 and/or 241 and the loop 232 and 242 shape(s) appropriate for a given clinical application. For example, when occluder 20 is formed from a polymer tube 25, straight loops 232 and 242 may be preferable because they will impart maximum stiffness to occluder 20. If the tube 25 is formed of a stiffer material, the angled slits 231 and/or 241 may provide a more desired stiffness to the occluder 20.

In one embodiment, the occluder 20 has loops according to FIGS. 2A-2D on one side and loops according to FIGS. 2E-2H on the other side. For example, occluder 20 may comprise loops 42 on the proximal side 40 and loops 232 on the distal side 30, or it may comprise loops 242 on the proximal side 40 and loops 32 on the distal side 30.

In one embodiment, for example as shown in FIG. 2H, each loop 242 and 232 has some amount of twist, i.e., when the loop is formed, the proximal side of the loop is radially offset with respect to the distal side of the loop. Loops 242 and/or 232, however, need not have any twist.

FIG. 2M, for example, illustrates an embodiment of the occluder with slits cut as illustrated in FIG. 2L. In this embodiment, neither loops 32 nor loops 42 are twisted. It will be apparent to one skilled in the art that any combination of twisted and untwisted loops may be used. Furthermore, an occluder can have any combination of loops with different bends and twists if desired.

In one embodiment, loops 32 (or 232) of distal side 30 are bent to form concave loops, while loops 42 (or 242) of proximal side 40 are flat (FIG. 11). In this embodiment, the outermost portions of loops 42 (or 242) of proximal side 40 oppose the outermost portions of the loops 32 (or 232) of the proximal side 30, as described in more detail below, thereby creating a desirable opposing force that secures the occluder 20 at its desired location in vivo. So configured, the opposing compressive forces exerted by sides 30 and 40 on the septum 12 following deployment of occluder 20 in vivo is advantageous in certain circumstances, such as closing certain kinds of PFOs. In another embodiment, loops 42 (or 242 of the proximal side 40 are bent, while loops 32 (or 232) of the distal side 30 are flat. In yet another embodiment, loops 42 (or 242) of the proximal side 40 and loops 32 (or 232) of the distal side 30 are bent.

Whatever the number and shapes of loops 32 and 42 (or 232 and 242), the loops 32 and 42 (or 232 and 242) may be of varied sizes to facilitate delivery of occluder 20, e.g. to improve collapsibility of the occluder 20 or to enhance its securement at the delivery site. For example, loops 32 and 42 (or 232 and 242) that are sized to better conform with anatomical landmarks enhance securement of the occluder 20 to the septum 12 in vivo. As indicated above, the cross-sectional dimensions of loops 32 and 42 (or 232 and 242) are determined by the thickness of tube 25 and the distance between adjacent slits 31 and 41 (or 231 and 241). The length of slits 31 and 41 (or 231 and 241) determines the size of loops 32 and 42 (or 232 and 242) and the radial extent of the deployed occluder 20. In at least some embodiments, each of distal side 30 and proximal side 40 has a diameter in the range of about 10 mm to about 45 mm, with the particular diameter determined by the size of the particular defect being treated. In particular embodiments, the diameter of distal side 30 will be different than that of proximal side 40 so as to better conform to the anatomy of the patient's heart.

According to one embodiment of the invention, the loops of the occluder are formed by struts as illustrated in FIG. 2B. Sections 91a, 91b, 92a, 92b, 93a, 93b, 94a, and 94b are of equal distance, being about ⅓ the length of distal side 30 (i.e., the distance between central tube 22 and end 39) of the tube 25. According to another embodiment of the invention, other lengths of sections can be used to produce advantageous results. In general, the longer the length of the hemispherical struts, such as half sections 91a, 91b, 94a, and 94b, the stiffer the occluder will be. The longer the length of the quarter (as shown) struts, such as half sections 92a, 92b, 93a, and 93b, the less stiff the occluder will be. In general, the hemispherical cut (one of the two) may be 20-40% of the overall length of the distal side (or proximal side) the tube. Specifically, the hemispherical cuts could be 40% of the overall length of the distal side (or proximal side) and then the quarter cut could be 20% of the overall length of the distal side (or proximal side) of the tube 25. Also, the lengths of the hemispherical cuts need not be the same. It may be advantageous to shorten one or the other side of the hemispherical cut based on a desired stiffness characteristic for a particular application of the occluder. In an alternative structure, the hemispherical cuts can be extended in a range up to 100% of the length of the distal side (or the proximal side) of the occluder, while still enabling the bow and twist of the struts.

As indicated previously and shown in FIGS. 2A-2H, distal side 30 and proximal side 40 of occluder 20 are connected by central tube 22. The central tube 22 is formed by the portion of tube 25 between the distal side 30 of tube 25, which contains slits 31, (or 231) and the proximal side 40 of tube 25, which contains slits 41 (or 241). Given that the central portion of tube 25 remains uncut during the cutting process, the central portion of the tube maintains its profile upon the application of forces F_{d} and Fₚ and does not bow and twist outward as the proximal and distal sides are adapted to do.

According to one embodiment, central tube 22 is straight, as illustrated in FIGS. 2D and 2H, where the central tube 22 is perpendicular to loops 32 and 42 (or 232 and 242). According to another embodiment of the invention, central tube 22 is positioned at an angle θ relative to the proximal side 40 of the occluder 20, as shown, for example, in FIGS. 5B and 11. The shape of central tube 22 included in a given occluder is, at least in part, determined by the nature of the aperture 18. An occluder having a straight central tube 22 is particularly suited to treat an anatomical anomaly including a perpendicular aperture, such as an ASD and certain PFOs. Often, however, anatomical anomalies, such as certain PFOs, have non-perpendicular apertures and are sometimes quite significantly non-perpendicular. An occluder having an angled central tube 22 is well-suited for treatment of such defects, such that the angle of the anatomical aperture 18 is more closely matched by the pre-formed angle θ of the occluder 20. Also, the length of central tube 22 can be varied depending on the anatomy of the defect being closed. Accordingly, the distal side 30 and proximal side 40 of occluder 20 are more likely to be seated against and minimize distortion to the septum 12 surrounding the aperture 18, as shown in FIG. 13. A well-seated occluder 20 is less likely to permit blood leakage between the right 11 and left 13 atria, and the patient into which the occluder 20 has been placed is, therefore, less likely to suffer embolisms and other adverse events.

Advantageously, angled central tube 22 also facilitates delivery of occluder 20 because it is angled toward the end of the delivery sheath. In at least some embodiments, the angle θ is about 0-45 degrees. To form the angle θ, proximal side 40 of the occluder 20 bends depending upon, among other factors, the material used to form occluder 20. Accordingly, depending upon design considerations, tip 44 and end 39 may be aligned with central tube 22 or perpendicular to proximal side 40 or some variation in between. One skilled in the art will be capable of determining whether a straight or angled central tube 22 is best suited for treatment of a given anatomical aperture 18 and the appropriate angle θ, typically in the range between about 30 and about 90 degrees. Sometimes, angles of about 0 degrees to about 30 degrees can be used in an oblique passageway such as a very long tunnel PFO. One skilled in the art will recognize that the concept of an angled central tube may be applied to septal occluders other than those disclosed herein.

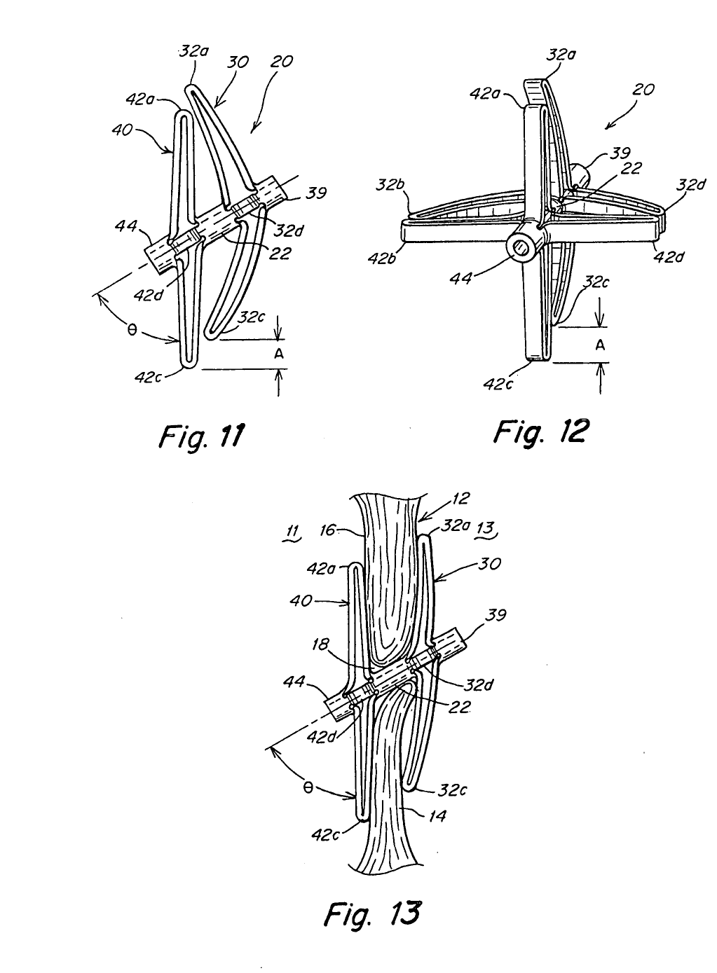

When central tube 22 is positioned at angle θ, distal side 30 and proximal side 40 of occluder 20 may be configured such that they are either directly opposing or, as shown in FIGS. 5B, 11 and 12, offset by distance A. One skilled in the art will, of course, recognize that the shape and arrangement of either or both of distal side 30 and proximal side 40 may be adjusted such that the compressive forces they apply are as directly opposing as possible. However, in some clinical applications, an occluder 20 having an offset of distance A may be particularly desirable. For example, as shown in FIGS. 5B, and 11-12, if the septum 12 surrounding aperture 18 includes a disproportionately thick portion (e.g. septum secundum 16 as compared to septum primum 14), the offset A may be used to seat occluder 20 more securely upon septum 12. Moreover, the offset A allows each of sides 30 and 40 to be centered around each side of an asymmetric aperture 18.

When a central tube 22 at angle θ is included in occluder 20, a marker is required to properly orient the occluder 20 in its intended in vivo delivery location. For example, a platinum wire may be wrapped around one of loops 32 or 42 (or one of loops 232 or 242) so as to permit visualization of the orientation of the occluder 20 using fluoroscopy. Alternatively, other types of markers may be used, e.g. coatings, clips, etc. As one skilled in the art would appreciate, the radiopaque marker could be blended in with the extrudate and thus provide visibility under fluoroscopy. As will be readily understood by one skilled in the art, the orientation of a non-symmetrical occluder 20 during delivery is of great importance. Of course, when a non-symmetrical occluder 20 is used, the periphery of the occluder 20 may be configured such that the clamping force applied by the proximal side 40 is directly opposed to that applied by the distal side 30.

Upon deployment in vivo (a process described in detail below), an occluder 20 according to the present invention applies a compressive force to the septum 12. Distal side 30 is seated against the septum 12 in the left atrium 13, central tube 22 extends through the aperture 18, and proximal side 40 is seated against the septum 12 in the right atrium 11. At least some portion of each of loops 32 and 42 (or 232 and 242) contacts septum 12. In particular embodiments, a substantial length of each of loops 32 and 42 (or 232 and 242) contacts septum 12. As illustrated in the representative Figures, the proximal side 40 and distal side 30 of occluder 20 overlap significantly, such that the septum 12 is “sandwiched” between them once the occluder 20 is deployed. According to at least some embodiments and depending upon the material used to form occluder 20, the loops 32 and 42 (or 232 and 242) provide both a radially-extending compressive force and a circumferential compressive force to septum 12. In these embodiments, the compressive forces are more evenly and more widely distributed across the surface of the septum 12 surrounding the aperture 18 and, therefore, provide the occluder 20 with superior dislodgement resistance as compared to prior art devices. As used in this application, “dislodgement resistance” refers to the ability of an occluder 20 to resist the tendency of the force applied by the unequal pressures between the right 11 and left 13 atria (i.e. the “dislodging force”) to separate the occluder 20 from the septum 12. Generally, a high dislodgement resistance is desirable.

Loops 32 and 42 (or 232 and 242) are also configured to minimize the trauma they inflict on the septum 12 surrounding aperture 18. Specifically, as indicated previously, the outer perimeter of loops 32 and 42 (or 232 and 242) may be rounded.

According to one embodiment of the invention, for example, as illustrated in FIGS. 2B-2D, the circumferential portions of loops 32 and 42 are thinner than the orthogonally-extending portions of loops 32 and 42; therefore, the center of the occluder 20 is stronger than its perimeter. Accordingly, outer perimeter of loops 32 and 42 of occluder 20 has a low compression resistance. As used in this application, “compression resistance” refers to the ability of an occluder 20 to resist the lateral compressive force applied by the heart as it contracts during a heartbeat. Generally, an occluder that resists compressive force, i.e. has high compression resistance, is undesirable because its rigid shape and arrangement may cause trauma to the septum 12, the right atrium 11, and/or the left atrium 13.

According to at least some embodiments of the present invention, occluder 20 further includes a catch system, generally indicated at 131, that secures the occluder 20 in its deployed state. The catch system 131, in general, maintains the shape and arrangement of loops 32 and 42 (or 232 and 242) of occluder 20, once the occluder 20 has been deployed. Catch system131 reduces and maintains the axial length of the occluder 20 so that occluder 20 maintains its deployed state, is secured in the aperture 18, and consistently applies a compressive force to septum 12 that is sufficient to close aperture 18. Catch system 131 is particularly advantageous when the occluder 20 is formed of a polymeric material, as previously described, because the polymeric occluder 20 may be deformed during delivery such that it may not fully recover its intended shape once deployed. By reducing and maintaining the axial length of occluder 20 once it has been deployed in vivo, catch system 131 compensates for any undesirable structural changes suffered by occluder 20 during delivery. In some embodiments, catch system 131 includes a ceramic material or a material selected from the group consisting of metals, shape memory materials, alloys, polymers, bioabsorbable polymers, and combinations thereof. In particular embodiments, the catch system may include nitinol or a shape memory polymer. Further, the catch system may include a material selected from the group consisting Teflon-based materials, polyurethanes, metals, polyvinyl alcohol (PVA), extracellular matrix (ECM) or other bioengineered materials, synthetic bioabsorbable polymeric scaffolds, collagen, and combinations thereof.

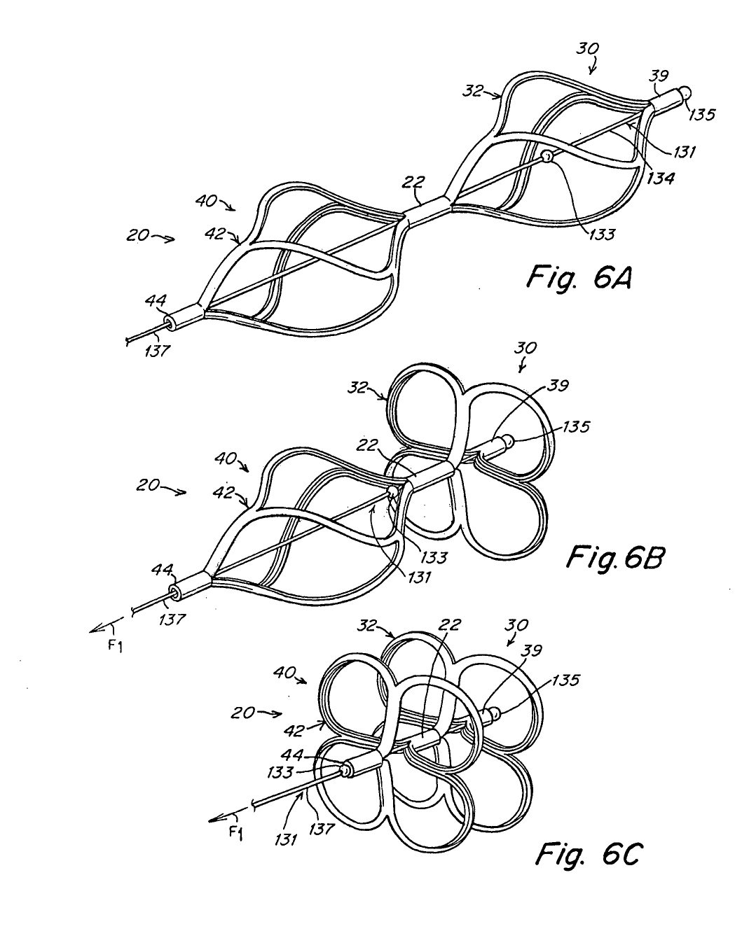

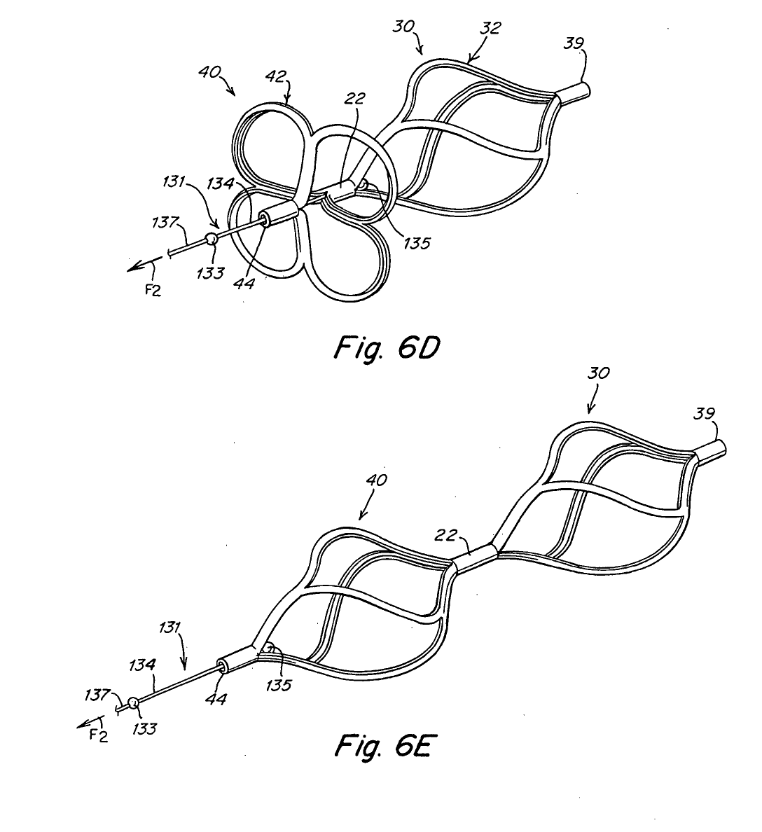

Catch system 131 may take a variety of forms, non-limiting examples of which are provided in FIGS. 6A-6E. For example, as shown in FIG. 6A, catch system 131 includes two catch elements, e.g., balls, 133 and 135, connected by wire 134. The catch system and catch element are preferably made of the same material as the occluder, although based on design selection, they could be made of the same or different material. In certain circumstances, it may be necessary to make them of different material. As illustrated in FIG. 6A, delivery string 137 is attached to ball 133 and is then extended through end 39, distal portion 30 of tube 25, central tube 22, proximal portion 40 of tube 25, and tip 44, such that ball 133 is located between central tube 22 and end 39 and ball 135 is located on the distal side of central tube 22. The function of catch system 131 is shown in FIGS. 6B-6E. Ball 133 is designed such that, upon the application of sufficient pulling force F₁, to delivery string 137, it passes through central tube 22 (FIG. 6B) and tip 44 (FIG. 6C). Ball 133 cannot reenter tip 44 or central tube 22 without the application of a sufficient, additional force. In this manner, ball 133 may be used to bring together the distal side 30 and the proximal side 40, thereby reducing and maintaining the axial length of occluder 20. Obviously, during the application of pulling force F₁, the tip 44 of occluder 20 must be held against an object, such as a delivery sheath. Ball 135 is designed such that, upon application of sufficient pulling force F₂ to delivery string 137, it passes through end 39 (FIG. 6D) and central tube 22 (FIG. 6E). The pulling force F₂ required to move ball 135 through end 39 and central tube 22 is greater than the pulling force F₁, required to move ball 133 through central tube 22 and tip 44. However, ball 135 cannot pass through tip 44. Thus, the application of sufficient pulling force F₂ to ball 135 releases distal side 30 and proximal side 40, as described in more detail below. It should be noted that while catch elements 133 and 135 are illustrated as spherical elements in FIGS. 6A-6E, catch elements 133 and 135 may take any suitable shape. For example, catch elements 133 and 135 may be conical. The narrow portions of conical catch elements 133 and 135 point toward tip 44 of proximal side 40. One possible mode of recovery or retrieval for this device is simply reversing the implantation procedure. Of course, other modes of recovery or retrieval are possible, some of which are described in this specification.

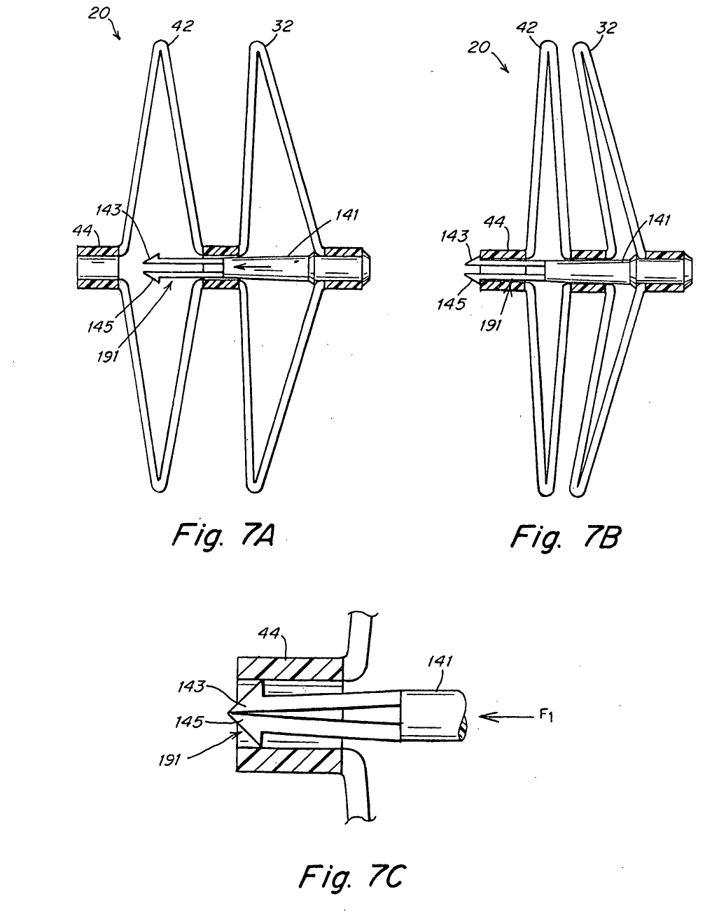

A different system for securing the device in the deployed state is shown in FIGS. 7A-7C. A locking mechanism 191 includes a hollow cylinder 141 having at least two half-arrows 143 and 145 located at its proximal end (FIG. 7A). Cylinder 141 enters tip 44 under application of pulling force F. to delivery string 137. As cylinder 141 enters tip 44, half-arrows 143 and 145 are forced together such that the diameter of the proximal end of cylinder 141 is reduced (FIG. 7B). Under continued application of pulling force F₁, half-arrows 143 and 145 pass through tip 44 and expand to their original shape and arrangement (FIG. 7C). Given that half-arrows 143 and 145 extend beyond the diameter of tip 44, the axial length of an occluder 20 including the locking mechanism 191 shown in FIGS. 7A-7C is maintained in its reduced state. If the implant needs to be removed or repositioned, the locking mechanism 191 shown in FIGS. 7A-7C may be released by moving half-arrows 143 and 145 together such that the diameter of the proximal end of cylinder 141 is smaller than that of tip 44 and cylinder 141 passes through tip 44. Cylinder 141 may then be withdrawn from tip 44.

One skilled in the art will recognize that catch system 131 may assume numerous configurations while retaining its capability to reduce and maintain the axial length of occluder 20 such that occluder 20 maintains its deployed state. For example, catch system 131 may include a threaded screw, a tie-wrap, or a combination of catch systems 131. Furthermore, catch system 131 may include multiple members that may provide a stepped deployment process. For example, catch system 131 as depicted in FIGS. 6A-6E may include three balls. In this configuration, one ball is used to secure the distal end 30 of occluder 20 and another ball is used to secure the proximal end 40 of occluder 20, and the third ball is secured to the distal end. Any suitable catch system 131 may be incorporated into any of the embodiments of occluder 20 described herein. One skilled in the art will be capable of selecting the catch system 131 suitable for use in a given clinical application.

Occluder 20 may be modified in various ways. According to some embodiments of the present invention, distal side 30 and/or proximal 40 side of occluder 20 may include a tissue scaffold. The tissue scaffold ensures more complete coverage of aperture 18 and promotes encapsulation and endothelialization of septum 12, thereby further encouraging anatomical closure of the septum 12. The tissue scaffold may be formed of any flexible, biocompatible material capable of promoting tissue growth, including but not limited to polyester fabrics, Teflon-based materials, ePTFE, polyurethanes, metallic materials, polyvinyl alcohol (PVA), extracellular matrix (ECM) or other bioengineered materials, synthetic bioabsorbable polymeric scaffolds, other natural materials (e.g. collagen), or combinations of the foregoing materials. For example, the tissue scaffold may be formed of a thin metallic film or foil, e.g. a nitinol film or foil, as described in United States Patent Publ. No. 2003/0059640 (the entirety of which is incorporated herein by reference). In those embodiments, where occluder 20 includes a tissue scaffold, the scaffold may be located on the outside the face of distal side 30 and proximal side 40 of the occluder, with an alternative of including scaffold also inside the face of distal side 30 and proximal side 40 of the occluder. Also, the tissue scaffold could be disposed against the tissue that is sought to be occluded, such as the septum 12 so that the proximity of the tissue scaffold and septum 12 promotes endothelialization. Loops 32 and 42, (or 232 and 242), can be laser welded, ultrasonically welded, thermally welded, glued, or stitched to the tissue scaffold to securely fasten the scaffold to occluder 20. One skilled in the art will be able to determine those clinical applications in which the use of tissue scaffolds and/or stitches is appropriate.

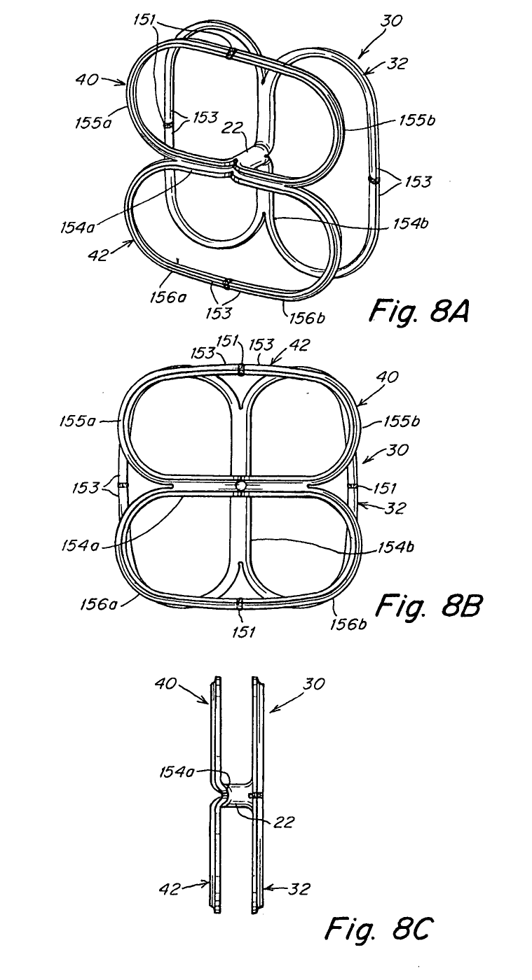

Occluder 20 may be further modified so that it lacks end 39 and tip 44, as shown in FIGS. 8A-8C, and, therefore, has a reduced septal profile. Such an occluder may be formed in several ways. For example, according to one embodiment, slits 31 and 41 are extended through end 39 and tip 44, respectively, of tube 25 during the cutting process. This cutting pattern produces struts 32 that deform during deployment to produce incomplete loops 32. One side of the device, facing the viewer as shown in FIG. 8A, is formed by slits 31 that extend along the tube 25 to varying lengths. The tube 25 is cut in half to form half sections 154a and 154b. The half sections 154a and 154b are further cut to a proximal distance from the end 39 into quarter sections 155a, 156a, 155b, and 156b. The ends of the quarter sections 155a and 155b are joined at “free” ends 153 to close the loop 32. Similarly, the free ends of quarter sections 156a and 156b may be joined by appropriate cutting, see FIG. 8b. The ends may be joined using any suitable connectors, e.g., 151, e.g., welds. One of skill in the art will recognize that the free ends 153 of loops 32 connected using other means, including but not limited to seams and bonds obtained by heat or vibration.

In the above embodiment, the slits in the quarter sections are run completely through the end of the tube 39. In an alternative embodiment, the end 39 may remain uncut, thereby eliminating the need for a weld to join the quarter sections together.

The embodiment illustrated in FIGS. 8A-8C depicts an occluder 20 in which both sides are formed according to the above-described design. Alternatively, an occluder 20 according to the present invention may include a hybrid structure, wherein one side is designed according to the embodiment shown in FIGS. 8A-8C and the other side is designed according to other types of structures disclosed in this application.

Occluder 20 may be prepared for delivery to an aperture 18 in any one of several ways. Slits 31 and 41 (or 231 and 241) may be cut such that tube 25 bends into its intended configuration following deployment in vivo. Specifically, slits 31 and 41 (or 231 and 241) may be cut to a thickness that facilitates the bending and formation of loops 32 and 42 (or 232 and 242). Upon the application of forces F_{d} and Fₚ, tube 25 bends into its intended deployed configuration. Alternatively and/or additionally, tube 25 formed of a shape memory material may be preformed into its intended configuration ex vivo so that it will recover its preformed shape once deployed in vivo. According to at least some embodiments, these preforming techniques produce reliable deployment and bending of occluder 20 in vivo. An intermediate approach may also be used: tube 25 may be only slightly preformed ex vivo such that it is predisposed to bend into its intended deployed configuration in vivo upon application of forces F_{d} and Fp.

An occluder 20 as described herein may be delivered to an anatomical aperture 18 using any suitable delivery technique. For example, distal side 30 and proximal side 40 of occluder 20 may be deployed in separate steps, or both distal side 30 and proximal side 40 of occluder 20 may be deployed in the same step. One delivery method will be described in detail herein.

When a patient has an implanted device made of a radiopaque bioabsorbable material, the position and orientation of the device can be monitored during the implantation procedure. At a later time, the device can be again viewed radiographically, and changes in its density or size resulting from bioabsorption, as well as in its position and/or orientation, can be assessed. This feature can be useful both for monitoring the health and recovery progress of patients, as well as for developing and improving the form of and materials used in future devices based on the observed results.

As shown in FIGS. 9A-9H, a delivery sheath 161 containing pusher sleeve 169 (shown in FIG. 9H) is used to deliver occluder 20 including the catch system 131 illustrated in FIGS. 6A-6E. Sheath 161 contains occluder 20 in its elongated, delivery form (FIG. 9A). As shown in FIG. 9B, delivery sheath 161 is first inserted into the right atrium 11 of the patient's heart. Sheath 161 is next inserted through aperture 18 located in the septum 12 (which, in this example, is a PFO tunnel) and into the left atrium 13 (FIG. 9C). Distal side 30 of occluder 20 is then exposed into the left atrium 13, as shown in FIG. 9D. Following deployment of distal side 30, pulling force F₁ is applied to delivery string 137 while pusher sleeve 169 is holding the occluder 20 in place such that ball 133 passes through the central tube 22, thereby securing distal side 30 into its deployed state (FIG. 9E). Sheath 161 is further withdrawn through the aperture 18 and into the right atrium 11, such that central tube 22 is deployed through the aperture 18 (FIG. 9F). Proximal side 40 of occluder 20 is then exposed into the right atrium 11 (FIG. 9G), and pulling force F₁ is again applied to delivery string 137 while pusher sleeve 169 is holding the occluder 20 in place such that ball 133 passes through tip 44, thereby securing the proximal side 40 into its deployed state (FIG. 9H). When properly deployed, occluder 20 rests within the aperture 18, and the distal side 30 and proximal side 40 exert a compressive force against septum primum 14 and septum secundum 16 in the left 13 and right 11 atria, respectively, to close the aperture 18, i.e. the PFO. When occluder 20 is properly deployed, delivery string 137 is detached from catch system 131, including balls 133 and 135 and a connecting member, and sheath 161 is then withdrawn from the heart. In the event occluder 20 is not properly deployed after performing the procedure described above, the occluder 20 may be recovered by reversing the steps of the above described delivery sequence.

In an alternative recovery technique, the occluder 20 may be recovered and repositioned by catch system 131 as shown in FIG. 10A-10D. Pusher sleeve 169 in sheath 161 is positioned against tip 44 of the occluder 20 in the right atrium 11 (FIG. 10A). Pulling force F₂ is applied to delivery string 137, such that ball 135 passes through end 39 and into central tube 22, thereby releasing distal side 30 from its deployed state (FIG. 10B). Force F₂ is again applied to delivery string 137 so that ball 135 subsequently passes through central tube 22, thereby releasing proximal side 40 from its deployed state (FIG. 10C). Delivery string 137 is then pulled further such that occluder 20, now in its elongated state, is retracted into sheath 161 (FIG. 10D). Following recovery of occluder 20, sheath 161 may be withdrawn from the heart and another occluder inserted in the desired delivery location as described above and shown in FIGS. 9A-9H.

One skilled in the art will recognize that the occluders described herein may be used with anti-thrombogenic compounds, including but not limited to heparin and peptides, to reduce thrombogenicity of the occluder and/or to enhance the healing response of the septum 12 following deployment of the occluder in vivo. Similarly, the occluders described herein may be used to deliver other drugs or pharmaceutical agents (e.g. growth factors, peptides). The anti-thrombogenic compounds, drugs, and/or pharmaceutical agents may be included in the occluders of the present invention in several ways, including by incorporation into the tissue scaffold, as previously described, or as a coating, e.g. a polymeric coating, on the tube(s) 25 forming the distal side 30 and proximal side 40 of the occluder 20. Furthermore, the occluders described herein may include cells that have been seeded within the tissue scaffold or coated upon the tube(s) 25 forming the distal side 30 and proximal side 40 of the occluder 20.

One skilled in the art will further recognize that occluders according to this invention could be used to occlude other vascular and non-vascular openings. For example, the device could be inserted into a left atrial appendage or other tunnels or tubular openings within the body.

The radiopaque bioabsorbable material described in present invention could be used to make devices for repairing, replacing, remodeling or closing intracardiac septal and atrial appendage defects, for sealing of a percutaneous puncture in a blood vessel or organ; stents, sutures and varies and orthopedic applications.

Having described certain embodiments, it should be apparent that modifications can be made without departing from the scope of the invention as defined by the appended claims. For example, certain materials have been stated, although other suitable materials could be used. In another example, a radiopaque bioabsorbable material made with one radiopaque agent blending with one bioabsorbable material have been described, although a mixture of radiopaque agents could be blended with one or a mixture of bioabsorbable material to make a radiopaque bioabsorbable material.

Having described preferred embodiments of the invention, it should be apparent that various modifications may be made without departing from the spirit and scope of the invention, which is defined in the claims below.

What is claimed is:

1. An occluder for a biological defect to be introduced into the body through the vasculature, the occluder including a structural member consisting essentially of a radiopaque bioabsorbable material, the radiopaque bioabsorbable material having a thickness between 500 and 750 microns, wherein the radiopaque bioabsorbable material comprises a blend of a bioabsorbable material and a radiopaque material, the radiopaque material having a linear attenuation coefficient greater than about 9 cm⁻¹, the radiopaque bioabsorbable material containing between 20 to 35 percent by weight of the radiopaque material.

2. The occluder of claim 1, wherein the bioabsorbable material is selected from a group consisting of polyglycolic acid, polylactic acid, poly caprolactone, poly (hyrodxybutyrate), poly(hydroxyvalerate), poly(sebacic acid-headecanoic acid anhydride), polyorthoester, polydioxanone, polygluconate, poly(amino acid), poly(alpha hydroxyl acid), and co-polymers thereof.

3. The occluder of claim 1, wherein the radiopaque material is tungsten in the form of a powder.

4. The occluder of claim 3, wherein the tungsten powder has a particle size in the range between about 0.5 to about 2.0 microns.

5. An occluder for a biological defect, including a structural member consisting essentially of a radiopaque bioabsorbable material, wherein the material comprises a blend of a bioabsorbable material and a radiopaque material, the bioabsorbable material having a molecular weight of at least 300,000, and the radiopaque material having a linear attenuation coefficient greater than about 9 cm⁻¹.

6. The occluder of claim 5, wherein the occluder has a proximal side and a distal side that cooperate to close the defect, and at least one of the proximal side or the distal side includes petals, and wherein the petals are formed by the structural member consisting essentially of a bioabsorbable radiopaque material.

7. The occluder of claim 5, wherein the occluder further comprises tissue scaffolding attached to the occluder.

8. The occluder of claim 5, wherein the bioabsorbable material is a bioabsorbable polymer.

9. The occluder of claim 8, wherein the bioabsorbable polymer is selected from a group consisting of polyglycolic acid, polylactic acid, poly caprolactone, poly (hyrodxybutyrate), poly(hydroxyvalerate), poly(sebacic acid-headecanoic acid anhydride), polyorthoester, polydioxanone, polygluconate, poly(amino acid), poly(alpha hydroxyl acid), and co-polymers thereof.

10. The occluder of claim 5, wherein the radiopaque material is tungsten in the form of a powder.

11. The occluder of claim 10, wherein the tungsten powder has a particle size in the range between about 0.5 and about 2.0 microns.

12. The occluder of claim 11, wherein the tungsten powder has a particle size in the range between 0.5 and 2.0 microns.

13. The occluder of claim 5, wherein the radiopaque bioabsorbable material has a weight percent of tungsten in the range between about 20 and 35 weight percent.

14. The occluder of claim 5, wherein the radiopaque, bioabsorbable material has a thickness between 500 and 750 microns.

15. The occluder of claim 5, wherein the occluder is made from a tube with slits that form petals when the tube changes from a delivery configuration to a deployed configuration.

16. The occluder of claim 15, wherein the occluder has a proximal side and a distal side that cooperate to close the defect and the proximal side includes proximal petals and the distal side includes distal petals.

17. The occluder of claim 16, wherein the occluder further comprises tissue scaffolding attached to at least one of the distal petals or the proximal petals.

18. The occluder of claim 16, wherein the occluder is a patent foramen ovale (PFO) occluder.

19. The occluder of claim 15, wherein the tube consists essentially of the radiopaque bioabsorbable material.

20. The occluder of claim 15, wherein the bioabsorbable material is a bioabsorbable polymer.

21. The occluder of claim 20, wherein the bioabsorbable material is selected from a group consisting of polyglycolic acid, polylactic acid, poly caprolactone, poly (hyrodxybutyrate), poly(hydroxyvalerate), poly(sebacic acid-headecanoic acid anhydride), polyorthoester, polydioxanone, polygluconate, poly(amino acid), poly(alpha hydroxyl acid), and co-polymers thereof.

22. The occluder of claim 15, wherein the radiopaque material is tungsten in the form of a powder.

23. The occluder of claim 22, wherein the tungsten powder has a particle size in the range between about 0.5 and about 2.0 microns.

24. The occluder of claim 23, wherein the tungsten powder has a particle size in the range between 0.5 and 2.0 microns.

25. The occluder of claim 5, wherein the radiopaque bioabsorbable material has a weight percent of tungsten in the range between about 20 and 35 weight percent.

26. The occluder of claim 5, wherein the radiopaque, bioabsorbable material has a thickness between 500 and 750 microns.

27. An occluder for a biological defect, including a structural member consisting essentially of a radiopaque bioabsorbable material, wherein the material comprises a blend of a bioabsorbable material and a radiopaque material, the bioabsorbable material having a molecular weight of at least 300,000, and the radiopaque material having a mass attenuation coefficient greater than about 1.2 cm²/gm.

28. The occluder of claim 27, wherein the occluder has a proximal side and a distal side that cooperate to close the defect, and at least one of the proximal side or the distal side includes petals, and wherein the petals are formed by the structural member consisting essentially of a bioabsorbable radiopaque material.

29. The occluder of claim 27, wherein the occluder further comprises tissue scaffolding attached to the occluder.

30. The occluder of claim 27, wherein the bioabsorbable material is a bioabsorbable polymer.

31. The occluder of claim 30, wherein the bioabsorbable polymer is selected from a group consisting of polyglycolic acid, polylactic acid, poly caprolactone, poly (hyrodxybutyrate), poly(hydroxyvalerate), poly(sebacic acid-headecanoic acid anhydride), polyorthoester, polydioxanone, polygluconate, poly(amino acid), poly(alpha hydroxyl acid), and co-polymers thereof.

32. The occluder of claim 27, wherein the radiopaque material is tungsten in the form of a powder.

33. The occluder of claim 32, wherein the tungsten powder has a particle size in the range between about 0.5 and about 2.0 microns.

34. The occluder of claim 33, wherein the tungsten powder has a particle size in the range between 0.5 and 2.0 microns.

35. The occluder of claim 27, wherein the radiopaque bioabsorbable material has a weight percent of tungsten in the range between about 20 and 35 weight percent.

36. The occluder of claim 27, wherein the radiopaque, bioabsorbable material has a thickness between 500 and 750 microns.

37. The occluder of claim 27, wherein the occluder is made from a tube with slits that form petals when the tube changes from a delivery configuration to a deployed configuration.

38. The occluder of claim 37, wherein the occluder has a proximal side and a distal side that cooperate to close the defect and the proximal side includes proximal petals and the distal side includes distal petals.

39. The occluder of claim 38, wherein the occluder further comprises tissue scaffolding attached to at least one of the distal petals or the proximal petals.

40. The occluder of claim 39, wherein the occluder is a patent foramen ovale (PFO) occluder.

41. The occluder of claim 37, wherein the tube consists essentially of the radiopaque bioabsorbable material.

42. The occluder of claim 41, wherein the bioabsorbable material is a bioabsorbable polymer.

43. The occluder of claim 42, wherein the bioabsorbable material is selected from a group consisting of polyglycolic acid, polylactic acid, poly caprolactone, poly (hyrodxybutyrate), poly(hydroxyvalerate), poly(sebacic acid-headecanoic acid anhydride), polyorthoester, polydioxanone, polygluconate, poly(amino acid), poly(alpha hydroxyl acid), and co-polymers thereof.

44. The occluder of claim 37, wherein the radiopaque material is tungsten in the form of a powder.

45. The occluder of claim 44, wherein the tungsten powder has a particle size in the range between about 0.5 and about 2.0 microns.

46. The occluder of claim 45, wherein the tungsten powder has a particle size in the range between 0.5 and 2.0 microns.

47. The occluder of claim 37, wherein the radiopaque bioabsorbable material has a weight percent of tungsten in the range between about 20 and 35 weight percent.

48. The occluder of claim 37, wherein the radiopaque, bioabsorbable material has a thickness between 500 and 750 microns.

49. A method of implanting an occluder for a biological defect, comprising:
providing the occluder, wherein the occluder has a structural member consisting essentially of a radiopaque bioabsorbable material, wherein the material comprises a blend of a bioabsorbable material and a radiopaque material, the bioabsorbable material having a molecular weight of at least about 300,000, and the radiopaque material having a linear attenuation coefficient greater than about 9 cm⁻¹,
inserting the occluder into a subject using a catheter, and
viewing a position and orientation of the device radiographically during implantation.

50. The method of claim 49, further comprising viewing the occluder radiographically at a number of different times after implantation and monitoring changes in the occluder due to bioabsorption of the material.

51. The method of claim 49, wherein the bioabsorbable materials is a bioabsorbable polymer selected from a group consisting of polyglycolic acid, polylactic acid, poly caprolactone, poly (hydroxybutyrate), poly(hydroxyvalerate), poly(sebacic acid-headecanoic acid anhydride), polyorthoester, polydioxanone, polygluconate, poly(amino acid), poly(alpha hydroxyl acid), and co-polymers thereof.

52. The method of claim 49, wherein the radiopaque agent is tungsten.

53. A method of making a radiopaque, bioabsorbable medical implant having a structural member formed of a blended radiopaque bioabsorbable material, made of a bioabsorbable polymer and a radiopaque agent, comprising:
selecting a biocompatible radiopaque agent for blending with a bioabsorbable polymer;
determining a concentration of said radiopaque agent in the radiopaque bioabsorbable material to attain a desired level of radiopacity;
identifying a desired initial criteria for a particular physical property of the radiopaque bioabsorbable material, wherein the physical property will vary as the material is bioabsorbed after implantation;
selecting the bioabsorbable polymer according to the desired initial criteria;
blending the selected radiopaque agent and the selected bioabsorbable polymer according to the determined concentration to form the blended material; and
forming the structural member using the blended material.

54. The method of claim 53, wherein the desired initial criteria is determined based on an expected rate of bioabsorption and an expected life of the implant.

55. The method of claim 53, wherein the physical property is molecular weight.

56. The method of claim 53, wherein the radiopaque agent is tungsten.

57. The method of claim 53, wherein the concentration of the radiopaque agent is between about 20 and 35 weight percent.

58. The method of claim 53, wherein the bioabsorbable polymer is selected from a group consisting of polyglycolic acid, polylactic acid, poly caprolactone, poly (hyrodxybutyrate), poly(hydroxyvalerate), poly(sebacic acid-headecanoic acid anhydride), polyorthoester, polydioxanone, polygluconate, poly(amino acid), poly(alpha hydroxyl acid), and co-polymers thereof.
